(19) United States
(12) Patent Application Publication
(10) Pub. No.: US 2026/0240792 A1
(43) Pub. Date: Aug. 20, 2026

(54) COMPOSITIONS FOR TREATING 5-HT2 CONDITIONS AND METHODS OF USING THE SAME

(71) Applicant: Board of Supervisors of Louisiana State University and Agricultural and Mechanical College, Baton Rouge, LA (US)

(72) Inventors: Timothy Foster, Slidell, LA (US); Diana Battaglia, Baton Rouge, LA (US)

(21) Appl. No.: 18/878,998

(22) PCT Filed: Jul. 3, 2023
(86) PCT No.: PCT/US2023/026845

§ 371 Date: Dec. 26, 2024

Related U.S. Application Data
(60) Provisional application No. 63/358,396, filed on Jul. 5, 2022.

(51) Int. Cl. A61K 31/137; A61K 9/00; A61K 9/08; A61K 47/38; A61P 27/02

(52) U.S. Cl. CPC A61K 31/137; A61K 9/0048; A61K 9/08; A61K 47/38; A61P 27/02

(57) ABSTRACT

Aspects of the invention are drawn to compositions and methods of treating a 5-HT2-mediated condition in a subject in need thereof. In embodiments, the 5-HT2-mediated condition is a 5-HT2A-mediated ocular condition. In some embodiments, the ocular condition is associated with decreased lacrimation or tear film production (e.g., dry eye), in certain embodiments, the ocular condition is associated with increased IOP (e.g., glaucoma).

This application claims the benefit of U.S. Provisional Application No. 63/358,396, filed on Jul. 5, 2022, the entire contents of which are incorporated herein by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

In general, the invention features compositions and methods for treating 5-HT2 responsive conditions.

### BACKGROUND OF THE INVENTION

Significant interest in the therapeutic application of 5-HT2 receptor ligands has developed, based upon evidence of therapeutic effects in a wide array of 5-HT2 responsive conditions (e.g., mitigating or improving conditions and disorders via 5-HT2 receptor target modulation), including psychiatric conditions, pain disorders, immunological conditions, and neurological conditions.

There is a need in the field for discovery and development of small molecule 5-HT2 receptor ligands with more desirable therapeutic, absorption, distribution, pharmacokinetic, and/or safety profiles.

### SUMMARY OF THE INVENTION

The invention features a method of treating a 5-HT2-mediated ocular condition in a subject in need thereof, the method including administering less often than once daily to an eye of the subject compound 1:

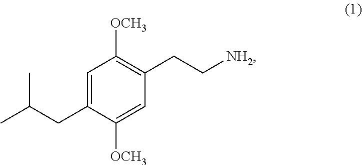 [SMILES: COc1cc(CC(C)C)c(OC)cc1CCN]

or a pharmaceutically acceptable salt thereof, in an amount effective to treat the ocular condition. In embodiments, the 5-HT2-mediated ocular condition is a 5-HT2A-mediated ocular condition. In some embodiments, the ocular condition is associated with decreased lacrimation or tear film production (e.g., dry eye). In certain embodiments, the ocular condition is associated with increased IOP (e.g., glaucoma).

The ocular condition can be the result of a side effect associated with another treatment undertaken by the subject. For example, the ocular condition can be decreased lacrimation or tear film production in a subject undergoing treatment with prostaglandins, alpha-adrenergic agonists, beta-adrenergic blocking agents, rho kinase inhibitors, or carbonic anhydrase inhibitors.

In certain embodiments, the ocular condition is IOP in a subject undergoing treatment with an ocular steroid.

In some embodiments, the ocular condition is associated with inflammation (e.g., conjunctivitis or keratitis).

In embodiments, the ocular condition is associated with pathological ocular neovascularization. For example, the pathological ocular neovascularization can be a corneal neovascularization or a choroidal neovascularization. The pathogenic ocular neovascularization being treated can be associated with macular degeneration, keratoconjunctivitis, conjunctivitis, diabetic retinitis, retinopathy of prematurity, polypoidal choroidal vasculopathy, ischemic proliferative retinopathy, retinitis pigmentosa, cone dystrophy, proliferative vitreoretinopathy, retinal artery occlusion, retinal vein occlusion, Leber's disease, retinal detachment, retinal pigment epithelial detachment, rubeosis iridis, corneal neovascularization, retinal neovascularization, choroidal neovascularization, retinochoroidal neovascularization, cancer, or a combination thereof. In embodiments, the ocular condition to be treated is macular degeneration. In some embodiments, the ocular condition to be treated is associated with scarring of the eye.

In any of the methods described herein, the administering can be topical administration, instillation in the conjunctival sac, intravitreal administration, subconjunctival administration, retrobulbar, intracameral, or sub-Tenon's administration. In embodiments, the administering is by eye drop or gel.

In any of the methods described herein, compound 1, or a pharmaceutically acceptable salt thereof, can be administered once every other day to once monthly to the eye of the subject. For example, compound 1, or a pharmaceutically acceptable salt thereof, can be administered to an eye of the subject once every 2 days, 3 days, 4 days, 5 days, 6 days, 7 days, 8 days, 9 days, 10 days, 11 days, 12 days, 13 days, or 14 days to the eye of the subject.

In an embodiment of any of the methods described herein, an eye drop or instillation comprising from about 0.005% to about 0.15% (w/w) of the compound 1, or a pharmaceutically acceptable salt thereof, is administered to the subject.

In an aspect, the invention features an aqueous pharmaceutical composition suitable for ocular administration comprising: (i) from about 0.005% to about 0.15% (w/w) of compound 1:

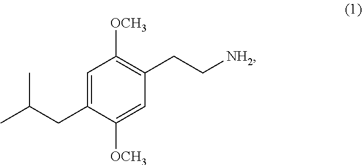 [SMILES: COc1cc(CC(C)C)c(OC)cc1CCN]

or a pharmaceutically acceptable salt thereof; and (ii) a buffer, wherein the aqueous pharmaceutical composition has a pH of between 5.0 and 7.0. For example, the buffer can be a phosphate, borate, or citrate buffer. In embodiments, the aqueous pharmaceutical composition further comprises a viscosity enhancer. In some embodiments, the aqueous pharmaceutical composition is free of preservatives.

Other objects and advantages of this invention will become readily apparent from the ensuing description.

### BRIEF DESCRIPTION OF THE FIGURES

FIG. 1 shows a schematic of a non-limiting, exemplary embodiment of assessment parameters.

FIG. 2 shows a schematic of a non-limiting, exemplary experimental design.

FIG. 3 shows a schematic of a non-limiting, exemplary embodiment of assessment parameters.

FIG. 4 shows charts (panels A, B, E) and graphs (panels C, D, F, G, H) of non-limiting, exemplary experimental data.

FIG. 5 shows a chart of Intraocular Pressure (IOP)-lowering glaucoma drugs from van der Valk R1, Webers C A, Schouten J S, Zeegers M P, Hendrikse F, Prins M H. Intraocular pressure-lowering effects of all commonly used glaucoma drugs: a meta-analysis of randomized clinical trials. Ophthalmology. 2005 July; 112(7):1177-85.

FIG. 6 shows charts (panels A, C) and graphs (panels B, D) of non-limiting, exemplary experimental data.

FIG. 7 shows charts (panels A, C, E) and graphs (panels B, D, F) of non-limiting, exemplary experimental data of Compound 1 effects on IOP.

FIG. 8 shows charts (panels A, C) and graphs (panels B, D) of non-limiting, exemplary experimental data of Compound 1 effects on tear film.

FIG. 9 shows a schematic of a non-limiting, exemplary embodiment of assessment parameters.

FIG. 10 shows charts (panels A, B) and a graph (panel C) of non-limiting experimental data for IOP.

FIG. 11 shows charts (panels A, B) and a graph (panel C) non-limiting experimental data of % change relative to initial data sets IOP.

FIG. 12 shows a graph (panel A) and chart (panel B) of non-limiting experimental data of changes in tears.

FIG. 13 shows a schematic of non-limiting examples of disease pathways.

FIG. 14 shows a schematic of non-limiting examples of serotonin linkages with disease.

FIG. 15 shows a schematic of non-limiting examples of serotonin linkages with dry eye.

FIG. 16 shows a schematic of non-limiting examples of serotonin linkages with glaucoma.

FIG. 17 shows a schematic of non-limiting examples of topical ocular delivery safety and tolerability.

FIG. 18 shows a schematic of non-limiting examples of 5-HT2AR in herpetic eye disease.

FIG. 19 shows images non-limiting examples of therapeutics in herpetic eye disease (panels A, B, C, D, E) and graphs (panels F, G, H) regarding the same.

FIG. 20 shows graphs of non-limiting examples of neurological effects.

FIG. 21 shows graphs of non-limiting experimental data of HSV-1 Viral Titer.

FIG. 22 shows drawings of healthy and damaged eye indications.

FIG. 23 shows graphs of non-limiting experimental IOP data during treatment.

FIG. 24 shows images of PC-12 Rat Neural Crest Cells subject to control and treatments with R-DOI.

FIG. 25 shows graphs of tear film, changes in tear film, and a schematic of lacrimal and meibomian glands.

FIG. 26 shows graphs of tear film production.

FIG. 27 shows schematics of pathways in the eye.

FIG. 28 shows non-limiting, experimental data of IL-17 concentration, and nonlimiting experimental flow cytometry data.

FIG. 29 shows graphs of non-limiting, experimental data of cytokine production.

FIG. 30 shows schematics of non-limiting examples of fibrosis pathways.

FIG. 31 shows a schematic of non-limiting examples of TGF-β signaling pathways.

FIG. 32 shows schematics and graphs of effects of TFG-β.

FIG. 33 shows schematics of non-limiting examples of fibrosis pathways.

FIG. 34 shows images of TGF-β transformed normal human fibroblasts to myofibroblasts.

FIG. 35 shows images of effects of DOI on transformation into myofibroblasts.

FIG. 36 shows images of human myofibroblasts.

FIG. 37 shows schematics and non-limiting experimental data of the effects of serotonin and 5-HT2A agonists on various indication and pathways.

FIG. 38 shows an image and graph of protein concentrations in rabbit aqueous humour.

### DETAILED DESCRIPTION OF THE INVENTION

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an”, and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.” Terms such as “a”, “an,” and “the” are not intended to refer to only a singular entity but include the general class of which a specific example can be used for illustration.

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b, and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the present invention in any appropriate manner.

As used herein, the term “standard of care” can refer to a diagnostic and/or treatment process for which a clinician follows for a certain type of patient, illness, or clinical circumstance. For example, a standard of care can refer to the ordinary level of skill and care that a clinician is expected to observe in providing clinical care to a patient. In embodiments, the standard of care can vary depending on the patient, the illness, or clinical circumstance. As used herein, “standard care practices” can refer to practices which are standard of care.

As used herein, the term “clinician” can refer to a person qualified in the clinical practice of medicine, psychiatry, or psychology. As used herein, the terms “clinician” and “practitioner” can be used interchangeably. For example, “clinician” can refer to a physician, a surgeon, a veterinarian, a physician assistant, a nurse, or a person practicing under the supervision thereof.

The terms “subject” and “patient,” as used herein, can include all members of the animal kingdom including, but not limited to, mammals, animals (e.g., cats, dogs, horses, swine, etc.) and humans.

The term “administration” or “administering” refers to a method of giving a dosage of a compound or pharmaceutical composition to a subject.

Aspects of the invention are drawn towards compounds and methods of treating 5-HT2 responsive conditions. The compounds can be useful for treating 5-HT2-responsive conditions.

The term “therapeutically effective amount,” as used herein, refers to an amount, e.g., pharmaceutical dose, effective in inducing an effect in a subject or in treating a subject having a condition or disorder described herein. It is also to be understood herein that a “therapeutically effective amount” can be interpreted as an amount giving a desired therapeutic and/or preventative effect, taken in one or more doses or in any dosage or route, and/or taken alone or in combination with other therapeutic agents. For example, in the context of administering a composition described herein that is used for the treatment of a disorder or condition, an effective amount of a compound is, for example, an amount sufficient to prevent, slow down, or reverse the progression of the disorder or condition as compared to the response obtained without administration of the compound.

As used herein, the terms “treat,” “treating,” or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (e.g., by reducing one or more symptoms of a 5-HT2 responsive condition). The term “therapeutic” includes the effect of mitigating deleterious clinical effects. The methods of the invention can be used as a primary prevention measure, i.e., to prevent a condition or to reduce the risk of developing a condition. Prevention refers to prophylactic treatment of a patient who can have fully developed a condition or disorder, but who is susceptible to, or otherwise at risk of, the condition. Thus, in the claims and embodiments, the methods of the invention can be used for therapeutic or prophylactic purposes.

### Serotonin 5-Hydroxytryptamine (5-HT) Receptors and Agonists

5-HT is known for its role as a neurotransmitter. Within the brain, 5-HT modulates a variety of behaviors including cognition, mood, aggression, mating, feeding, and sleep (Nichols and Nichols, 2008) (Bear, Connors, and Paradiso, Neuroscience Exploring the Brain, Wolter Kluwer, 2016). The effects of 5-HT are mediated through interactions at seven different families of receptor proteins (5-HT receptors), comprised of 14 different subtypes, consisting of 13 G-protein coupled receptors and one ligand-gated ion channel. For example, 5-HT1A, 5-HT1B, 5-HTID, 5-HT1E, 5-HT2A, 5-HT2B, 5-HT4, and 5-HT5A are G-protein-coupled neurotransmitter receptors for 5-HT (Bear et al.). Of all the serotonin receptors, the 5-HT2A receptor, which is known to couple to the Gαq effector pathway (Roth et al., 1986), has been linked to complex behaviors. There is a high level of expression of 5-HT2A receptors within the frontal cortex, with localization to the apical dendrites of cortical pyramidal cells (Willins et al., 1997), and further expression at lower levels throughout the brain (Nichols and Nichols, 2008). These receptors have been shown to participate in processes such as cognition and working memory, have been implicated in affective disorders such as schizophrenia, and have been shown to mediate the primary effects of hallucinogenic drugs (Nichols, 2004).

In addition, many peripheral tissues express 5-HT2A receptors. Within the vasculature, 5-HT2A receptors are known to modulate vasoconstriction (Nagatomo et al., 2004). Its role in other tissues such as mesangial cells of the kidney, fibroblasts, liver, and lymphocytes remains less defined, but has been linked to cellular proliferation and differentiation.

The presence of 5-HT2A receptor mRNA (along with the mRNAs of other serotonin receptor subtypes) has been found in tissues involved in the immune response (Stefulj et al., 2000). The role of 5-HT2A receptors in inflammatory processes, however, is unclear, with only a few published and inconsistent reports. For example, blockade of 5-HT2A receptor function with the selective antagonist sarpogrelate has been reported to decrease expression of proinflammatory markers (Marconi et al., 2003; Akiyoshi et al., 2006), and conversely has also been reported to increase expression of proinflammatory markers (I to et al., 2000). Using high non-pharmacologically relevant drug doses (lowest dose was 25 μM), a 5-HT2 receptor specific agonist, 1-(2,5-dimethoxy-4-iodophenyl)-2-aminopropane (DOI; the racemic form), was alleged to repress IL-1β expression and production of TNF-α due to lipopolysaccharide stimulation through 5-HT2A receptor activation (Cloez-Tayarani et al., 2003).

Two other studies reported that DOI acting at 5-HT2 receptors partially blocked LPS and cytokine stimulated nitrite accumulation using a cocktail of TNF-α and INF-γ in C6 glioma cells and reported an IC₅₀ value of 8±3 nM. (Miller et al., 1997; Miller and Gonzalez, 1998). In earlier reports, the synthesis of TNF-α in response to LPS stimulation of toll-like receptors was reported to be inhibited by 5-HT through 5-HT2 receptors in monocytes (Arzt et al., 1991). The use of ketanserin as the antagonist to block 5-HT2 receptor activation in many of these studies to indicate 5-HT2A receptor involvement is problematic, because ketanserin has only weak selectivity (~20-fold) for 5-HT2A receptors over 5-HT2C receptors, and has a high affinity for α₁-adrenergic receptors and is equipotent at blocking histamine H1 receptors, which are known to regulate inflammatory processes. Ketanserin cannot be used to discriminate reliably between the effects of agonists acting at 5-HT2A or 5-HT2C receptors.

### 5-HT2A Agonists

In embodiments, the term “agonist” can refer to a compound that binds to a receptor and elicits a response. In embodiments, the term “antagonist” can refer to a compound that interferes with or inhibits a biological response by binding to and blocking a receptor. For example, the term “5-HT2A agonists” can be any compound that increases the activity of a 5-hydroxytryptamine 2A receptor. Examples of such agonists comprise DOI (=)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2′S,4′S)-(+)-9,10-didehydro-6-methylergoline-8β-(trans-2,4-dimethylazetidide); 2C-BCB (4-Bromo-3,6-dimethoxybenzocyclobuten-1-yl)methylamine; and lysergic acid diethylamide (LSD).

Without wishing to be bound by theory, complex actions of a compound at the receptor can be dose dependent, such that some doses of an agonist can have antagonist activity, or some doses of an antagonist can have agonistic activity. For example, the receptor agonistic activity of a compound can decrease responses at the receptor in certain conditions, thereby resulting in an elicited biological response.

### 5-HT2A Conditions and Indications

As used herein, the term “condition” can refer to a state of being or a health status of a patient or subject. As used herein, the term “disease” can refer to a condition of patient, subject, or a part thereof that impairs normal functioning and can be distinguished by signs and/or symptoms. As used herein, the term “symptom” can refer to evidence of disease or condition experienced by a patient or subject. As used herein, the terms “condition”, “disorder”, and “disease” can be used interchangeably. As used herein, the term “indication” can refer to a condition or disease which makes a treatment or procedure advisable or a sign or circumstance which points to or shows the cause, pathology, treatment, or outcome of an attack of disease.

As used herein, the term “5-HT2 condition” (or any variation thereof) can refer to any condition that can be affected by the engagement, modification, modulation and/or binding of the 5-HT2 receptor (5-HT2R). In embodiments, a 5-HT2 condition comprises a 5-HT2A condition. For example, a 5-HT2A condition can be affected by a 5-HT2A agonist and/or a 5-HT2A antagonist. As used herein, the term “5-HT2A condition” can refer to condition that can be affected by the engagement, modification, modulation and/or binding of the 5-HT2A receptor (5-HT2AR). In embodiments, the terms “5-HT2A condition” and “5-HT2A-mediated condition” can be used interchangeably. In embodiments, “5-HT2A-conditions” can comprise “5-HT2-mediated ocular conditions”. As used herein, the term “5-HT2-mediated ocular condition” can refer to an ocular condition which can be mediated by a 5-HT2 receptor. In embodiments, the 5-HT2-mediated ocular condition comprises a 5-HT2A-mediated ocular condition. As used herein, the term “5-HT2A-mediated ocular condition” can refer to an ocular condition which can be mediated by a 5-HT2A receptor.

In embodiments, compositions and methods described herein can be administered to a subject experiencing a 5-HT2A-mediated ocular condition. As used herein, the term “ocular condition” can refer to any condition connected with or relating to eyes or vision. In embodiments, the ocular conditions described herein can be 5-HT2A-mediated ocular conditions. In embodiments, the 5-HT2A-mediated ocular condition can comprise dry eye, decreased lacrimation, decreased tear production, increased intraocular pressure (IOP), glaucoma, a side effect of a treatment, inflammation, conjunctivitis, keratitis, pathological ocular neovascularization, macular degeneration, keratoconjunctivitis, diabetic retinitis, retinopathy of prematurity, polypoidal choroidal vasculopathy, ischemic proliferative retinopathy, retinitis pigmentosa, cone dystrophy, proliferative vitreoretinopathy, retinal artery occlusion, retinal vein occlusion, Leber's disease, retinal detachment, retinal pigment epithelial detachment, rubeosis iridis, corneal neovascularization, retinal neovascularization, choroidal neovascularization, retinochoroidal neovascularization, cancer, scarring of the eye, or a combination thereof.

In embodiments, compositions and methods described herein can be administered to a subject experiencing indications of a 5-HT2A-mediated ocular condition. In embodiments, indications of a 5-HT2A-mediated ocular condition can comprise dry eye, dysfunction tear syndrome, aqueous deficiency, goblet cell deficiency, meibomian gland dysfunction, tear film disorders, keratoconjunctivitis sicca, glaucoma, increased age, Sjorgen's syndrome, graft vs host disease, contact lens usage, corneal nerve desensitivity, laser eye surgery, lens surgery, refractive index surgery, ocular procedures, us of antihistamine eye drops, use of ophthalmic drug preservatives (e.g. BAK), eyelid disorders (e.g. extropion and entropion), infection, computer screen usage, rheumatoid arthritis, lupus, scleroderma, vitamin A deficiency, thyroid disorders, Parkinson's disease, smoking, exposure to pollution, exposure to wind, exposure to dry air climate, radiation therapy, or a combination thereof.

In embodiments, compositions and methods described herein can be administered to a subject experiencing decreased lacrimation. Indications of decreased lacrimation can comprise subjects administered steroids, eye drops comprising benzalkonium chloride (BAK), antihistamines, decongestants, hormone replacement therapies, antidepressants, high blood pressure medications, beta blockers, birth control, pain relievers/NSAIDS, GI proton pump inhibitors, anti-psychotic medications, chemotherapies, isotretinoin/retinoic acids, Accutane, anticholinergic drugs, prostaglandins, alpha-adrenergic agonists, beta-adrenergic blocking agents, carbonic anhydrase inhibitors, or any combination thereof.

### Compounds

Aspects of the invention are drawn to compounds and compositions which can engage the 5-HT2A receptor. In embodiments, the compounds which engage the 5-HT2A receptor comprise 5-HT2A receptor agonists.

Unless specifically stated, a compound can comprise isomers. The term “isomer” can refer to stereoisomers and/or geometric isomers of the inventive polymers, e.g., cis- and trans-isomers, R- and S-enantiomers, diastereomers, (D)-isomers, (L)-isomers, the racemic mixtures thereof, as well as “head-to-tail” and “tail-to-tail” configurational isomers.

In embodiments, compounds of the invention and methods of using the same can comprise structures according to:

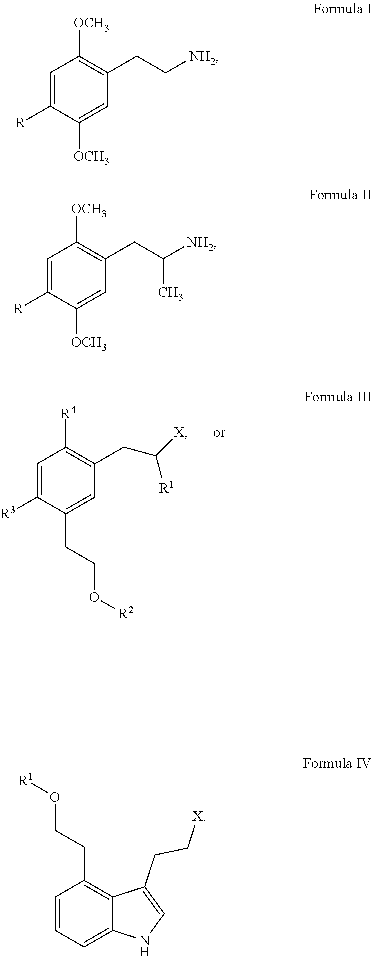

In embodiments the structures of Formulas I-IV can comprise: X is NH₂, NH(CH₃), N(CH₃)₂, or N(CH₃)₃; R¹ is H or CH₃; R² is H or CH₃; R³ is H, halogen, trifluoromethyl, CN, optionally substituted C₁₋₈ alkyl, optionally substituted C₂₋₈ alkenyl, optionally substituted C₁₋₈ alkoxy, and C₁-C₈ alkylthio; and R⁴ is H, OH, OCH₃, or OCH₂CH₃.

In embodiments, Formula (III) can comprise: X is NH₂, NH(CH₃), N(CH₃)₂, or N(CH₃)₃; R¹ is H or CH₃; R² is H or CH₃; R³ is H, halogen, trifluoromethyl, CN, optionally substituted C₁₋₈ alkyl, optionally substituted C₂₋₈ alkenyl, optionally substituted C₁₋₈ alkoxy, and C₁-C₈ alkylthio; and R⁴ is H, OH, OCH₃, or OCH₂CH₃. In an embodiment of formula (III), R³ is selected from bromo, iodo, CN, —CH₃, —CH₂CH₃, —CH(CH₃)₂, —CH₂CH(CH₃)₂, —CH₂CH₂CH(CH₃)₂, —CH₂CH₂CH₂CH₃, —C(CH₃)₃, —CH₂C(CH₃)₃; —OCH(CH₃)₂, —OC(CH₃)₃, and C₁-C₈alkylthio, or a group selected from:

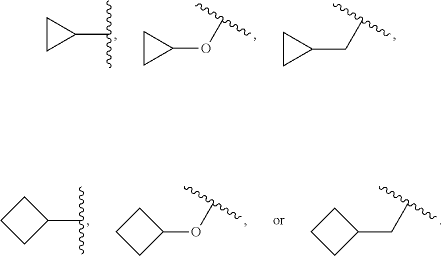

In embodiments, for example, compounds described herein can comprise structures according to:

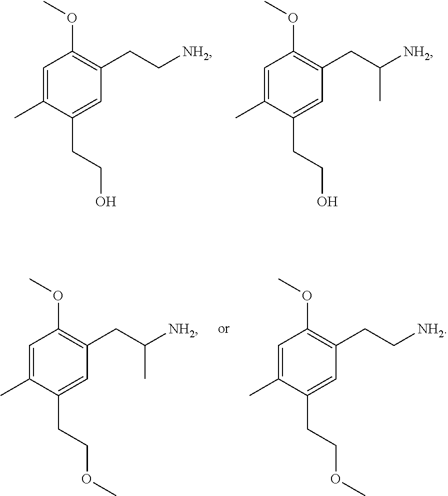 [SMILES: COc1cc(C)c(CCO)cc1CCN ; COc1cc(C)c(CCO)cc1CC(C)N ; COCCc1cc(CC(C)N)c(OC)cc1C ; "or" ; COCCc1cc(CCN)c(OC)cc1C]

In embodiments the structures of Formulas I-IV can comprise: R¹ is H or CH₃; X is N(R²)(R³) or N(R²)(R³)(CH₃); and each of R² and R³ are, independently, selected from —CH₃, —CH₂CH₃, and —CH(CH₃)₂, —CH₂CH(CH₃)₂.

For example, compounds described herein can comprise structures according to:

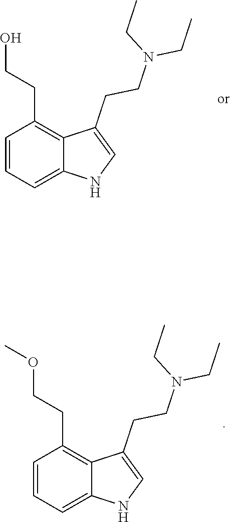

In embodiments, the “R” group of Formulas I-IV comprise a branched alkyl group or a cyclic alkyl group. For example, the compounds described can comprise the structure according to:

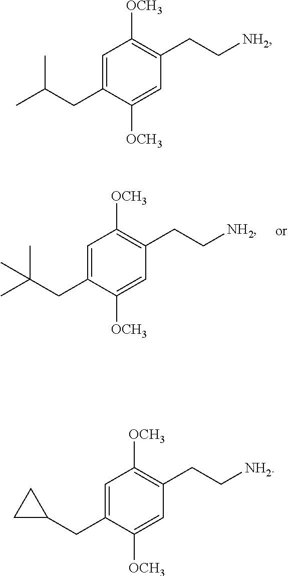 [SMILES: COc1cc(CC(C)C)c(OC)cc1CCN ; COc1cc(CC(C)(C)C)c(OC)cc1CCN ; COc1cc(CC2CC2)c(OC)cc1CCN]

In embodiments, the “R” group of Formulas I-IV comprise a halogen or an alkyl group. For example, the compound can comprise a structure according to:

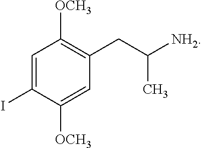

As used herein, 2,5-Dimethoxy-4-iodoamphetamine (DOI) can be described by a structure according to:

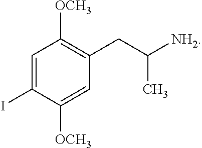

In certain embodiments, DOI can be the (R) isomer of DOI (R-DOI). As used herein, the terms “R-DOI” and “ELE-02” can be used interchangeably.

Aspects of the invention are drawn towards pharmaceutically acceptable salts of compounds described herein and methods of using the same. As used herein, the term “pharmaceutically acceptable salt” refers to those salts of the compounds described herein that are suitable for pharmaceutical use. Pharmaceutically acceptable salts are well known in the art. For example, pharmaceutically acceptable salts are described in: Berge et al., J. Pharmaceutical Sciences 66:1 19, 1977 and in Pharmaceutical Salts: Properties, Selection, and Use, (Eds. P. H. Stahl and C. G. Wermuth), Wiley VCH, 2008. The salts can be prepared in situ during the final isolation and purification of the compounds described herein or separately, e.g., by reacting the free base of the compound with a suitable organic acid or inorganic acid.

In embodiments, pharmaceutically acceptable salts can comprise hydrochloride.

Described herein, substituents of compounds of the present disclosure are disclosed in groups or in ranges. It is intended that the present disclosure include each and every individual sub-combination of the members of such groups and ranges. For example, the term alkyl can be described as “C1-8 alkyl”. “C1-8 alkyl” can refer to methyl, ethyl, C3 alkyl, C4 alkyl, C5 alkyl, C6 alkyl, C7 alkyl, and C8 alkyl. Herein, a phrase of the form “optionally substituted X” (e.g., optionally substituted alkyl) can be equivalent to “X, wherein X is optionally substituted” (e.g., “alkyl, wherein the alkyl is optionally substituted”). It is not intended to mean that the feature “X” (e.g., alkyl) per se is optional.

As used herein, the terms “alkyl” and the prefix “alk-” are inclusive of both straight chain and branched chain groups and of cyclic groups, i.e., cycloalkyl, and combinations thereof. Cyclic groups can be monocyclic or polycyclic and can have from 3 to 6 ring carbon atoms, inclusive. Exemplary cyclic groups include cyclopropyl, cyclobutyl, cyclopentyl, and cyclohexyl groups. The C1-8 alkyl group can be substituted or unsubstituted. Exemplary substituents include alkoxy, aryloxy, sulfhydryl, alkylthio, arylthio, halide, hydroxyl, fluoroalkyl, perfluoralkyl, cyano, nitrilo, NH-acyl, amino, aminoalkyl, disubstituted amino, C2-7 heterocyclyl, quaternary amino, hydroxyalkyl, carboxyalkyl, and carboxyl groups. C1-8 alkyls include, without limitation, methyl; ethyl; n-propyl; isopropyl; cyclopropyl; cyclopropylmethyl; cyclopropylethyl; n-butyl; iso-butyl; sec-butyl; tert-butyl; cyclobutyl; cyclobutylmethyl; cyclobutylethyl; n-pentyl; cyclopentyl; cyclopentylmethyl; cyclopentylethyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; and cyclohexyl.

“C2-8 alkenyl” refers to a branched or unbranched hydrocarbon group containing one or more double bonds and having from 2 to 8 carbon atoms. A C2-8 alkenyl can include monocyclic or polycyclic rings, in which each ring can have from three to six members. The C2-8 alkenyl group can be substituted or unsubstituted. Exemplary substituents include alkoxy, aryloxy, sulfhydryl, alkylthio, arylthio, halide, hydroxyl, fluoroalkyl, perfluoralkyl, cyano, nitrilo, NH-acyl, amino, aminoalkyl, disubstituted amino, quaternary amino, hydroxyalkyl, carboxyalkyl, and carboxyl groups. C2-8 alkenyls include, without limitation, vinyl; allyl; 2-cyclopropyl-1-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3-dimethyl-3-butenyl; 1,3-dimethyl-3-butenyl; 1,1-dimethyl-3-butenyl and 2,2-dimethyl-3-butenyl. In embodiments the C2-8 alkenyl has a cis configuration around the double bond.

“C2-7 heterocyclyl” refers to a stable 5- to 7-membered monocyclic or 7- to 14-membered bicyclic heterocyclic ring which is saturated, partially unsaturated, or unsaturated (aromatic), and which comprises 2 to 7 carbon atoms and 1, 2, 3, or 4 heteroatoms independently selected from the group consisting of N, O, and S, and including any bicyclic group in which any of the herein-defined heterocyclic rings is fused to a benzene ring. The heterocyclyl group can be substituted or unsubstituted. Exemplary substituents include alkoxy, aryloxy, sulfhydryl, alkylthio, arylthio, halide, hydroxy, fluoroalkyl, perfluoralkyl, cyano, nitrilo, NH-acyl, amino, aminoalkyl, disubstituted amino, quaternary amino, hydroxyalkyl, carboxyalkyl, and carboxyl groups. The nitrogen and sulfur heteroatoms can be oxidized. The heterocyclic ring can be covalently attached via any heteroatom or carbon atom which results in a stable structure, e.g., an imidazolinyl ring can be linked at the ring-carbon atom positions or at the nitrogen atom. A nitrogen atom in the heterocycle can be quaternized. For example, when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, beta-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. For example, 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. For example, 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

“C6-12 aryl” refers to an aromatic group having a ring system comprised of carbon atoms with conjugated x electrons (e.g., phenyl). The aryl group has from 6 to 12 carbon atoms. Aryl groups can comprise monocyclic, bicyclic, or tricyclic rings, in which each ring can comprise five or six members. The aryl group can be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

“C7-14 alkaryl” refers to an alkyl substituted by an aryl group (e.g., benzyl, phenethyl, or 3,4-dichlorophenethyl) having from 7 to 14 carbon atoms.

“C3-10 alkheterocyclyl” refers to an alkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

“C1-8 heteroalkyl” refers to a branched or unbranched alkyl, alkenyl, or alkynyl group having from 1 to 8 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl can comprise monocyclic, bicyclic, or tricyclic rings, in which each ring can comprise three to six members. The heteroalkyl group can be substituted or unsubstituted. Exemplary substituents include alkoxy, aryloxy, sulfhydryl, alkylthio, arylthio, halide, hydroxyl, fluoroalkyl, perfluoralkyl, cyano, nitrilo, NH-acyl, amino, aminoalkyl, disubstituted amino, quaternary amino, C2-7 heterocyclyl, hydroxyalkyl, hydroxyalkyl, carboxyalkyl, and carboxyl groups. Examples of C1-8 heteroalkyls include, without limitation, methoxymethyl and ethoxyethyl.

“Halide” refers to bromine, chlorine, iodine, or fluorine.

“Fluoroalkyl” refers to an alkyl group that is substituted with one or more fluorine atoms.

“Carboxyalkyl” refers to a chemical moiety with the formula —(R)—COOH, wherein R is selected from C1-8 alkyl, C2-7 heterocyclyl, C6-12 aryl, C7-14 alkaryl, C3-10 alkheterocyclyl, or C1-8 heteroalkyl.

“Hydroxyalkyl” refers to a chemical moiety with the formula —(R)—OH, wherein R is selected from C1-8 alkyl, C2-7 heterocyclyl, C6-12 aryl, C7-14 alkaryl, C3-10 alkheterocyclyl, or C1-8 heteroalkyl.

“Alkoxy” refers to a chemical substituent of the formula-OR, wherein R is selected from C1-8 alkyl, C2-7 heterocyclyl, C6-12 aryl, C7-14 alkaryl, C3-10 alkheterocyclyl, or C1-8 heteroalkyl.

“Aryloxy” refers to a chemical substituent of the formula-OR, wherein R is a C6-12 aryl group.

“Alkylthio” can refer to a chemical substituent of the formula-SR, wherein R is selected from C1-8 alkyl, C2-7 heterocyclyl, C6-12 aryl, C7-14 alkaryl, C3-10 alkheterocyclyl, or C1-8 heteroalkyl.

“Arylthio” refers to a chemical substituent of the formula-SR, wherein R is a C6-12 aryl group.

“Quaternary amino” refers to a chemical substituent of the formula —(R)—N(R′)(R″)(R′″)+, wherein R, R′, R″, and R′″ are each independently an alkyl, alkenyl, alkynyl, or aryl group. R can comprise an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

“Acyl” refers to a chemical moiety with the formula R—C(O)—, wherein R is selected from C1-8 alkyl, C2-7 heterocyclyl, C6-12 aryl, C7-14 alkaryl, C3-10 alkheterocyclyl, or C1-8 heteroalkyl.

### Pharmaceutical Compositions

Pharmaceutical compositions formulated for ocular delivery can provide an active ingredient in the form of droplets of a solution and/or suspension. Such formulations can be prepared, packaged, and/or sold as aqueous solutions, optionally sterile, including active ingredient, and can conveniently be administered using a dropper. Such formulations can further include one or more additional ingredients including, but not limited to a buffering agent, a surface-active agent, and/or a preservative such as methylhydroxybenzoate. Droplets provided by this route of administration can have an average diameter in the range from about 0.1 nm to about 200 nm. In embodiments, the composition is formulated as an aqueous pharmaceutical composition suitable for ocular administration. For example, the compositions described herein can be provided in the form of a drop, such as an eye drop or instillation, and the pharmaceutical formulation can further contain antioxidants and/or known agents for the treatment of eye diseases. In embodiments, the composition can be formulated as an eye gel. In embodiments, the aqueous pharmaceutical composition can comprise about 0.001% to about 0.5% (w/w) of a compound and/or pharmaceutically acceptable salts thereof described herein. In further embodiments, the composition can comprise a buffer. For example, the buffer can be at a pH of between about 5.0 to about 7.0. In embodiments, the buffer can comprise a phosphate buffer, a borate buffer, or a citrate buffer. In further embodiments, the pharmaceutical composition can comprise a viscosity enhancer. In embodiment, the pharmaceutical composition can be free of preservatives. The compounds described herein can be formulated into pharmaceutical compositions using techniques and procedures well known in the art (see, e.g., Ansel Introduction to Pharmaceutical Dosage Forms, Fourth Edition 1985, 126).

### Methods and Dosing

Provided herein are methods of using a compound or pharmaceutical composition described herein to treat 5-HT2 responsive conditions in a subject. Methods of treating 5-HT2 responsive conditions include administering to a subject in need thereof a therapeutically effective amount of a compound or pharmaceutical composition of the invention.

Surprising findings of the invention comprise that the compounds described here (e.g. R-DOI) can exhibit an extended half-life following administration to the eye, and so can be dosed less often than once daily. For example, a compound or pharmaceutically acceptable salt thereof described herein can be administered about once every day, about once every two days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every month, about once every two months, about once every 3 months, about once every 6 months, or about once every year.

In certain embodiments, the composition administered to the eye can comprise about 0.001%, about 0.002%, about 0.003%, about 0.004%, about 0.005%, about 0.010%, about 0.015%, about 0.020%, about 0.025%, about 0.030%, about 0.035%, about 0.040%, about 0.045%, about 0.050%, about 0.055%, about 0.060%, about 0.065%, about 0.070%, about 0.075%, about 0.080%, about 0.085%, about 0.090%, about 0.10%, about 0.125%, about 0.15%, about 0.20%, about 0.30%, about 0.40%, about 0.50%, about 0.75%, about 1%, about 2.5%, about 5%, about 10%, about 25%, or about 50% w/w of a compound described herein.

The exact amount of the compound or composition required for therapeutic effect can vary from subject to subject, depending on the species, age, weight, and general condition of the subject, the severity of the disease, the composition, its mode of administration, its mode of activity, and the like. Pharmaceutical compositions in accordance with the present disclosure can be formulated in a dosage form suitable for ease of administration and uniformity of dosage. It will be understood, however, that the total daily usage of the compositions of the present disclosure will be decided by the attending physician within the scope of sound medical judgment. The specific therapeutically effective level for a subject will depend upon a variety of factors including the inflammatory disorder being treated and the severity thereof; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts.

Compositions described herein can be administered to subjects, such as human patients or, alternatively, to other mammals, such as domesticated animals, cats, dogs, mice, or rats.

Compositions described herein can be administered ocularly.

A compound of the invention can be administered in a therapeutically effective amount below a threshold amount that elicits psychoactive effects (e.g., an amount that results in the desired therapeutic effect, e.g., within the therapeutic window between a dose sufficient to elicit a therapeutic effect and a dose that elicits a psychoactive effect (for example, about a ten-fold or greater selective index)). In some embodiments, a therapeutically effective amount of the compound that does not induce a psychoactive effect can be 0.1% (1 mg/mL) or less administered as a topical 50 μL drop to each eye or an equivalent total dose of less than 100 μg/kg. In some embodiments, a therapeutically effective amount of the compound in a topical that does not induce a psychoactive effect can be about 0.01%, about 0.02%, about 0.03%, about 0.04%, about 0.05%, about 0.06%, about 0.07%, about 0.08%, or about 0.09%. In some embodiments, a therapeutically effective amount of the compound that does not induce a psychoactive effect can be about 0.5 μg/kg, about 1 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 96 μg/kg, about 97 μg/kg, about 98 μg/kg, or about 99 μg/kg. In some embodiments, a therapeutically effective amount of the compound that does not induce a psychoactive effect can be about 0.5 μg/kg to about 1 μg/kg, about 1 μg/kg to about 5 μg/kg, about 5 μg/kg to about 10 μg/kg, about 10 μg/kg to about 20 μg/kg, about 20 μg/kg to about 30 μg/kg, about 30 μg/kg to about 40 μg/kg, about 40 μg/kg to about 50 μg/kg, about 50 μg/kg to about 60 μg/kg, about 60 μg/kg to about 70 μg/kg, about 70 μg/kg to about 80 μg/kg, about 80 μg/kg to about 90 μg/kg, or about 90 μg/kg to about 100 μg/kg.

In certain embodiments, compositions in accordance with the present disclosure can be administered at dosage levels sufficient to deliver from about 0.0001 μg/kg to about 1 mg/kg, from about 0.01 μg/kg to about 500 μg/kg, from about 0.1 μg/kg to about 400 μg/kg, from about 0.5 μg/kg to about 30 μg/kg, from about 0.01 μg/kg to about 10 μg/kg, from about 0.1 μg/kg to about 10 μg/kg, or from about 1 μg/kg to about 25 μg/kg, of subject body weight per day, one or more times a day, to obtain the desired therapeutic effect. The desired dosage can be delivered three times a day, two times a day, once a day, every other day, every third day, every week, every two weeks, every three weeks, or every four weeks. In some embodiments, the compound is administered at a frequency of one to three times per week (e.g., once per week, twice per week, three times per week, four times per week, five times per week, six times per week, seven times per week, or more, e.g., once daily, twice daily, three times daily, etc.). In some embodiments, the compound is administered intermittently, e.g., every other day, every other week, once per month, etc. In certain embodiments, the desired dosage can be delivered using multiple administrations (e.g., two, three, four, five, six, seven, eight, nine, ten, eleven, twelve, thirteen, fourteen, or more administrations).

Compositions described herein can be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Pharmaceutical compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that improve their bioavailability, reduce and/or modify their metabolism, inhibit their excretion, and/or modify their distribution within the body.

It will further be appreciated that compounds or compositions utilized in combination can be administered together in a single composition or administered separately in different compositions. Further, agents utilized in combination will be utilized at levels that do not exceed the levels at which they are utilized individually. In some embodiments, the levels utilized in combination will be lower than those utilized individually.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Method for Increasing Ocular Tear Production without Adversely Affecting Intraocular Pressure Through 5-HT2A Receptor Modulation: Treatment for Dysfunctional Tear Syndromes and Associated Tear Film Disorders

We have unexpectedly found that certain 5-HT2A receptor agonists at certain topical ocular doses increase tear production, while simultaneously lowering intraocular pressure. That is in contrast to the native 5-HT2A receptor agonist, serotonin, which can cause decreased tear production and development of dry eye (Chhadva, Priyanka et al. “Human Tear Serotonin Levels Correlate with Symptoms and Signs of Dry Eye.” Ophthalmology vol. 122, 8 (2015): 1675-80). In addition, drugs that increase serotonin availability and thereby 5-HT2A receptor binding (SSRIs) can also cause decreased tear production (Zhang et al., Selective Serotonin Reuptake Inhibitors Aggravate Depression-Associated Dry Eye Via Activating the NF-κB Pathway. Invest Ophthalmol Vis Sci. 2019 Jan. 2; 60 (1): 407-419; Koçer et al. Dry Eye Related to Commonly Used New Antidepressants. J Clin Psychopharmacol. 2015 August; 35(4): 411-3; Rakofsky et al., Dry Those Crying Eyes: The Role of Depression and Antidepressants in Dry Eye Disease. J Clin Psychopharmacol. 2021 May-Jun. 1; 41 (3): 295-303; and Dankis et al., Acute Inhibitory Effects of Antidepressants on Lacrimal Gland Secretion in the Anesthetized Rat. Invest Ophthalmol Vis Sci. 2021 Jun. 1; 62 (7): 8). Some of the investigated 5-HT2A receptor agonists did not have this effect. In addition, some exhibited a transient effect, whereas others exhibited prolonged tear production following a single dose.

Described herein, we determined a topically applied dosage of the 5-HT2A agonist that was ocularly tolerable without eliciting an adverse behavioral response. The ability of these drugs to induce increased tear production has implications for a number of aqueous production deficiencies that result in dysfunctional tear syndrome, as well as in combination drug therapies that can counteract these drug's tear production lowering effects.

### Non-Limiting Indications

Dry Eye
Dysfunctional Tear Syndrome
Aqueous Deficiency
Goblet Cell Deficiency
Meibomian Gland Dysfunction
Tear Film Disorders
Keratoconjunctivis Sicca
Glaucoma (in some embodiments, mixed with other IOP lowering compositions that inherently reduce tear production)
Aged populations where tear production naturally decreases, especially women over 50
Allergic Eye
Sjogren's syndrome
Graft vs host disease impact
Contact lens use
Corneal nerve desensitivity
Laser Eye Surgery
Lens Surgery
Refractive Surgery
Ocular Procedures
Antihistamine Eye Drops (again, in some embodiments, mixing with these drops to counteract their decreased tear production)
Certain Ophthalmic Drug Preservatives (BAK)
Eyelid Disorders-ectropion and entropion
Infection
Computer Screen Usage
Other diseases/conditions that can cause decreased tear production:
Rheumatoid arthritis
Lupus
Scleroderma
Graft vs host disease
Vitamin A deficiency
Thyroid Disorders
Parkinson's Disease
Smoking
Pollution
Windy Environment
Dry Air Climate
Radiation Therapy
Can supplement/Counteract other Drugs that lower tear production:
Antihistamines
Decongestants
Hormone replacement therapies-estrogen with or without progestin
Antidepressants (here any of the SSRIs have this effect, which is counterintuitive for the use of agonists in this instance)
High Blood Pressure Medications
Beta Blockers
Birth Control
Pain Relievers/NSAIDS
GI Proton Pump Inhibitors
Antipsychotic Meds
Chemotherapies, including that used in eye, like Cytoxan
Isotretinoin/retinoic acids in dermatological treatments
Accutane
Anticholinergic drugs for COPD or urinary incontinence

Non-limiting Advantages: All intraocular pressure lowering drugs have an inherent property of reducing tear production. However, topical ocular application of certain 5-HT2A agonists had both IOP lowering effects and increased tear production. With certain 5-HT2A agonist drugs, this increased tear production was maintained for an extended period of time following dosing.

### Example 2

### Non-Limiting, Exemplary Findings Discussed Herein

The API R-DOI HCl was formulated within an ophthalmic eye drop with BSS and 0.5% carboxymethylcellulose (CMC) at percentages of 0.1% and lower with a pH of 7.0.

R-DOI in acute single dosing and repeated dosing trials did not exhibit any acute ocular toxicity, irritability or adverse ocular reactions.

A systemic and behavioral threshold concentration for delivery was determined to be a delivery of at or greater than 100 total micrograms per animal. This total was delivered as a 50 μL drop of a 0.1% R-DOI ophthalmic solution to both eyes.

At 0.1% concentrations of R-DOI delivered to both eyes, only slight CNS associated behavioral effects were noted. However, increased respiration and heart rate were noted, indicating a systemic effect. The pupils of the eye also exhibited miosis or contraction of the pupil. Thus, without wishing to be bound by theory, miosis can be a local ocular reaction, rather than a CNS effect, because, generally, 5-HT2A agonists produce pupillary dilatation, a CNS effect.

Without wishing to be bound by theory, this was noted as a marker of adverse drug activity. Noted effects observed were cleared by one hour post administration.

No adverse events, including ocular, systemic, behavioral or otherwise were noted for any other dose administered: 0.01%, 0.05%, or 0.075%. Therefore, a dose greater than 0.075% or at 0.1% R-DOI can be the threshold at which effects begin to be observed. Multiple repeated dosing of 0.01% and 0.05% dosages did not cause any adverse events.

Administration of R-DOI decreased intraocular pressure with a single dose.

Administration of R-DOI increased tear production as measured by Schirmer's test.

### Preparation of Ophthalmic Solution of R-DOI HCl for Animal Ocular Studies:

### Calculations and Conversions of R-DOI HCl:

| R-DOI HCl | | MW 357.62 | |
|---|---|---|---|
| weight/volume | % conc. | Molar | |
| 1 g/100 mL | 1% | 27.96 | mM |
| 100 mg/100 mL | 0.10% | 2.796 | mM |
| 50 mg/100 mL | 0.05% | 1.398 | mM |
| 10 mg/100 mL | 0.01% | 0.2796 | mM |

The 0.1% stock was used in ocular pK studies and was utilized as the high concentration for beginning acute ocular toxicity and behavioral studies.

### Formulation of R-DOI Ophthalmic Eye Drops:

A 5 mL stock of 0.1% of R-DOI HCl in BSS with 0.5% carboxymethylcellulose (CMC) was made with 5 mg of R-DOI HCl. pH was at 7.0

Dilutions of Other Concentrations were Made from Stock as Follows:

| Stock: 5 mg/ml | | | |
|---|---|---|---|
| DILUTION | 0.1% | 2.796 mM | |
| 3:4 | 0.075% | 2.097 | mM |
| 1:2 | 0.05% | 1.398 | mM |
| 1:10 | 0.01% | 279.6 | μM |
| 1:5 | 0.02% | 559.3 | μM |

We can determine the ocular dose of R-DOI

HCl in this ophthalmic formulation that:
1) Did not produce behavioral effects.
2) Did not produce adverse systemic effects.
3) Was tolerated by the eye without adverse ocular effects, including absence of:
a. Apparent Ocular Irritability
b. Induction of Inflammation
c. Reduction of Corneal Barrier Integrity
d. Dry Eye or Excessive Tearing
e. Induction of Neovascularization
f. Induction of Scleral Reddening

### Overview of Experimental Layout:

| | Formu- | | Deliv- | #Eyes/ | Total | Obser- | Total |
|---|---|---|---|---|---|---|---|
| Dosage | lation | pH | ery | animal | Delivered | vation | Time |
| 0.01% | BSS | 7.0 | 50 μL/ | Both | 100 μL | See | 2 h |
| | 0.5% | | eye | (2) | | Listed | |
| | CMC | | | | | Table | |
| 0.05% | BSS | 7.0 | 50 μL/ | Both | 100 μL | See | 2 h |
| | 0.5% | | eye | (2) | | Listed | |
| | CMC | | | | | Table | |
| 0.075% | BSS | 7.0 | 50 μL/ | Both | 100 μL | See | 2 h |
| | 0.5% | | eye | (2) | | Listed | |
| | CMC | | | | | Table | |
| 0.10% | BSS | 7.0 | 50 μL/ | Both | 100 μL | See | 2 h |
| | 0.5% | | eye | (2) | | Listed | |
| | CMC | | | | | Table | |
| 0% | BSS | 7.0 | 50 μL/ | Both | 100 μL | See | 2 h |
| Con- | 0.5% | | eye | (2) | | Listed | |
| tro1 | CMC | | | | | Table | |

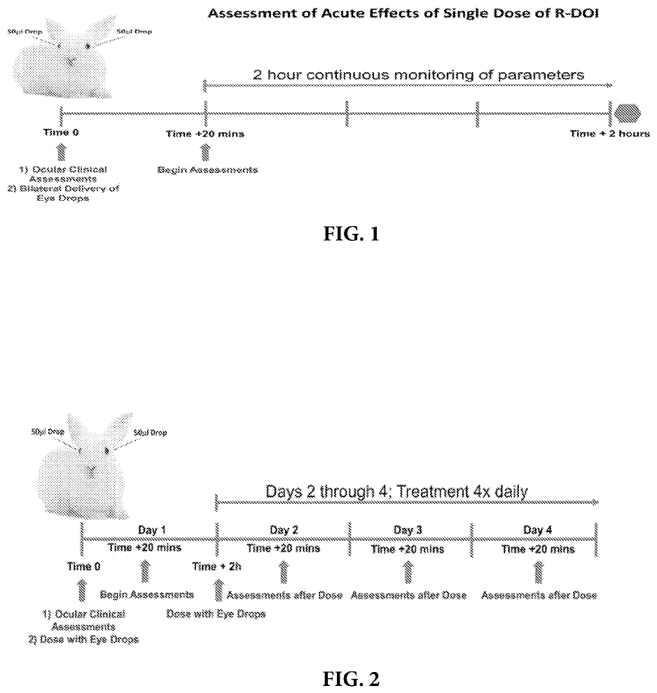

Parameters Assessed during 2 hour Observation Window (see, for example, FIG. 1):

### Observed Parameters:

### CNS:

head twitch
head bob
spasms
listing

### Systemic:

relative increased heart rate
relative increased respiration
short-term weight change

### Ocular:

pupil dilation/contraction
epiphora
intraocular pressure

### Behavioral:

grooming
response to auditory stimuli
lethargy
disorientation
aggression

### Animal Weight and Dosing:

| | | | R-DOI | | | |
|---|---|---|---|---|---|---|
| | | | HCl | | | |
| | | | (μg) | R-DOI | R-DOI | R-DOI |
| Animal | Weight | Treat- | dose/ | HCl | dose/ | dose |
| # | (kg) | ment | animal | μg/kg | animal | μg/kg |
| E1 | 2.82 | BSS; | 0 | 0.000 | 0 | 0 |
| | | 0.5% | | | | |
| | | CMC | | | | |
| E2 | 2.76 | BSS; | 0 | 0.000 | 0 | 0 |
| | | 0.5% | | | | |
| | | CMC | | | | |
| E3 | 2.88 | 0.1% | 100 | 34.72 | 89.80 | 31.18 |
| | | R-DOI; | | | | |
| | | BSS; | | | | |
| | | 0.5% | | | | |
| | | CMC | | | | |
| E4 | 2.70 | 0.1% | 100 | 37.04 | 89.80 | 33.26 |
| | | R-DOI; | | | | |
| | | BSS | | | | |
| | | 0.5% | | | | |
| | | CMC | | | | |
| E5 | 3.06 | 0.01% | 10 | 3.268 | 8.980 | 2.935 |
| | | R-DOI; | | | | |
| | | BSS; | | | | |
| | | 0.5% | | | | |
| | | CMC | | | | |
| E6 | 2.82 | 0.01% | 10 | 3.546 | 8.980 | 3.1844 |
| | | R-DOI; | | | | |
| | | BSS; | | | | |
| | | 0.5% | | | | |
| | | CMC | | | | |

### Outcomes:

Control Group (E1/E2; 0% drug): No observable changes; Animals were relaxed in cage grooming; All Normal Behaviors; No CNS, Ocular Behavioral or Systemic Changes. Response to Auditory Stimulation Normal.

Low Dose (E5/E6; 0.01% drug): No observable changes; Animals were relaxed in cage grooming; All Normal Behaviors; No CNS, Ocular Behavioral or Systemic Changes. Animals Eating and Grooming during observation. Response to Auditory Stimulation Normal.

### High Dose (0.1% Drug):

### Rabbit E3:

20 minutes post: Lethargy Rear of Enclosure; Increased breathing and Heartrate.
27 minutes post: Looking better; still lethargic. Miosis obvious (pupil contracted). Eyes glassy
35 minutes post: Heavy Breathing; Rapid Heartrate; Lethargy; Not as Responsive
39 minutes post: Possible Ear twitch; Miosis continues; Glassy stare
43 minutes post: Not very responsive to auditory stimuli; Glassy eyes; listing on side of cage 46 minutes post: Looking better; breathing more slowly/normally
50 minutes post: Sitting up and grooming; looking more normal
57 minutes post: Relaxed but still breathing faster than normal; Pupils near normal.
1 hour to 2 hour post: Regained Responsiveness; Breathing Normal; Pupils Normal

### Rabbit E4:

20 minutes post: Increased breathing and Heartrate; Still Responsive.
23 minutes post: Looking better; Grooming
33 minutes post: Slight heavy breathing
35 minutes post: Change in focus; Glassy Stare; Miosis
37 minutes post: Largely OK; Alert; Responsive; Grooming
42 minutes post: Lying down; Increased breathing; Sprawled out in Enclosure; Glossy eye; Miosis
44 minutes post: Up and Running in cage
45 minutes post: Head twitch?
47 minutes post: Normal behavior; Pupils more normal; Grooming
49 minutes post: Doing very well; Grooming; Moving around Enclosure;
51 minutes post: Sprawled out in enclosure; Eyes staring; slight miosis
53 minutes post: Seems to be OK; Normal Respiration; But sprawled out in enclosure
58 minutes post: All seems OK; Animal Upright; Pupils Normal; Breathing normal
1 hour to 2 hour post: Regained Responsiveness; Breathing Normal; Pupils Normal

Assessment: During the 2 hour observation windows, there were no adverse or abnormal events observed for rabbits treated with control or 0.01% R-DOI HCl as indicated. For animals treated with 0.1% R-DOI HCl, there was an onset of symptoms observed at the 20-minute assessment point that continued on and off until approximately 1 hour post treatment. There were minimal to no head bobs/twitches observed indicatory of CNS based effects. It was noted that there was a slight head and ear twitch observed once for each rabbit. However, these animals are housed individually in interconnected enclosures, and it cannot be sure that their responses were not to stimuli by other animal's activities. If there were any CNS effects they were minor, although the glassy eyes, vacant stare and miosis indicate that some CNS effects can have been occurring. Without wishing to be bound by theory, miosis can be a local ocular effect, rather than a CNS effect because 5-HT2A agonists in the CNS generally produce pupil dilation, not miosis. The observable adverse effects were systemic in nature, in the obvious increased respiration and heart rates. Overall, the assessment was that 0.01% was tolerated, while the 0.1% can represent the threshold dose due to systemic responses following treatment. Observable effects were absent at about 1 hour post treatment and this normalcy continued through the 2-hour observation window.

Non-Limiting, Exemplary Dose Finding Study for Range at which Adverse Effects are Observed:

Two studies were performed to determine at what dose between 0.01% and 0.1% were behavioral and/or systemic effects observed. 50 μL doses at 0.01%, 0.05%, and 0.075% per eye applied and assessments were made as indicated herein.

| | | | R-DOI | | | |
|---|---|---|---|---|---|---|
| | | | HCl | | | |
| | | | (μg) | R-DOI | R-DOI | R-DOI |
| Animal | Weight | Treat- | dose/ | HCl | dose/ | dose |
| # | (kg) | ment | animal | μg/kg | animal | μg/kg |
| E1 | 2.82 | BSS; | 0 | 0.000 | 0 | 0 |
| | | 0.5% | | | | |
| | | CMC | | | | |
| E2 | 2.76 | BSS; | 0 | 0.000 | 0 | 0 |
| | | 0.5% | | | | |
| | | CMC | | | | |
| E3 | 2.88 | 0.05% | 50 | 17.36 | 44.90 | 15.59 |
| | | R-DOI; | | | | |
| | | BSS; | | | | |
| | | 0.5% | | | | |
| | | CMC | | | | |
| E4 | 2.70 | 0.05% | 50 | 18.52 | 44.90 | 16.63 |
| | | R-DOI; | | | | |
| | | BSS; | | | | |
| | | 0.5% | | | | |
| | | CMC | | | | |
| E5 | 3.06 | 0.01% | 10 | 3.268 | 8.980 | 2.935 |
| | | R-DOI; | | | | |
| | | BSS; | | | | |
| | | 0.5% | | | | |
| | | CMC | | | | |
| E6 | 2.82 | 0.01% | 10 | 3.546 | 8.980 | 3.184 |
| | | R-DOI; | | | | |
| | | BSS; | | | | |
| | | 0.5% | | | | |
| | | CMC | | | | |

| | | | R-DOI | | | |
|---|---|---|---|---|---|---|
| | | | HCl | | | |
| | | | (μg) | R-DOI | R-DOI | R-DOI |
| Animal | Weight | Treat- | dose/ | HCl | dose/ | dose |
| # | (kg) | ment | animal | μg/kg | animal | μg/kg |
| E7 | 2.88 | BSS; | 0 | 0.000 | 0 | 0 |
| | | 0.5% | | | | |
| | | CMC | | | | |
| E8 | 2.94 | BSS; | 0 | 0.000 | 0 | 0 |
| | | 0.5% | | | | |
| | | CMC | | | | |
| E9 | 2.96 | 0.075% | 75 | 25.34 | 67.35 | 22.75 |
| | | R-DOI; | | | | |
| | | BSS; | | | | |
| | | 0.5% | | | | |
| | | CMC | | | | |
| E10 | 2.74 | 0.075% | 75 | 27.37 | 67.35 | 24.58 |
| | | R-DOI; | | | | |
| | | BSS; | | | | |
| | | 0.5% | | | | |
| | | CMC | | | | |
| E11 | 3.14 | 0.075% | 75 | 23.89 | 67.35 | 21.45 |
| | | R-DOI; | | | | |
| | | BSS; | | | | |
| | | 0.5% | | | | |
| | | CMC | | | | |
| E12 | 2.86 | BSS | 0 | 0.000 | 0 | 0 |
| | | 0.5% | | | | |
| | | CMC | | | | |

### Outcomes:

Control Group (E1/E2 and E7/E8 and E12; 0% drug): No observable changes; Animals were relaxed in cage grooming; All Normal Behaviors; No CNS, Ocular Behavioral or Systemic Changes. Response to Auditory Stimulation Normal.

Low Dose (E5/E6; 0.01% drug): No observable changes; Animals were relaxed in cage grooming; All Normal Behaviors; No CNS, Ocular Behavioral or Systemic Changes. Animals Eating and Grooming during observation. Response to Auditory Stimulation Normal.

Mid Dose (E3/E4; 0.05% drug): No observable changes; Animals were relaxed in cage grooming; All Normal Behaviors; No CNS, Ocular Behavioral or Systemic Changes. Animals Eating and Grooming during observation. Response to Auditory Stimulation Normal.

High Dose (E9/E10/E11; 0.075% drug): Animals were relaxed in cage grooming; All Normal Behaviors; No CNS, Ocular Behavioral or Systemic Changes. Animals Eating and Grooming during observation. Response to Auditory Stimulation Normal. Slight Miosis can be observed in E10 and E11 approximately 30 minutes post treatment. No other indications or changes.

Assessment: During the 2 hour observation windows, there were no adverse or abnormal events observed for rabbits treated with control, 0.01%, or 0.05% R-DOI HCl as indicated. For animals treated with 0.075% R-DOI HCl, there were no adverse CNS or systemic events observed. Slight but transient miosis was evident in 2 animals that resolved to normal within 15 minutes of initial observation. It is noted that miosis can be present when there is outflow of aqueous humor and reduction of IOP. Given other data and the activity of R-DOI at reducing IOP, this effect can be a marker of direct ocular effects, rather than an indicator of CNS effects. Without wishing to be bound by theory, it does however represent a marker of this drug's ocular activity. Given the combined studies, the threshold dose for inducing systemic effects and minimal undesirable events can be between 0.075% and 0.1% R-DOI HCl, with the only observable effects to date at 0.1% R-DOI applied to both eyes at 50 ul of delivery per eye for a total of 100 ug ocular delivery per animal. Tested levels below this dose produced no readily perceivable effects.

### Repeated Dosing of R-DOI and Assessment of Behavioral Effects:

The application of most topical eye drops requires repeatedly dosing up to 9 times per day in order to maintain efficacy across the course of waking hours and have efficacious levels available during sleeping hours. The effects of repeated ocular dosing of R-DOI were assessed in a similar manner as described herein, with the exception that doses of 0.01% or 0.05% were applied repeatedly as outlined herein:

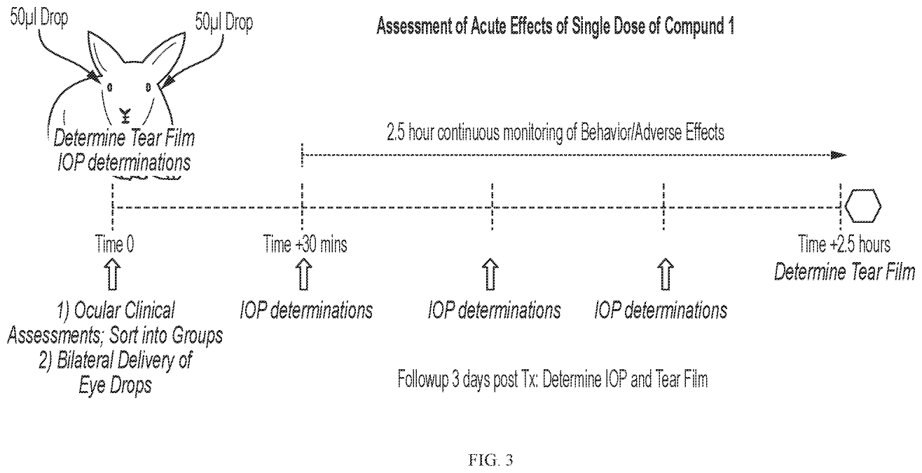

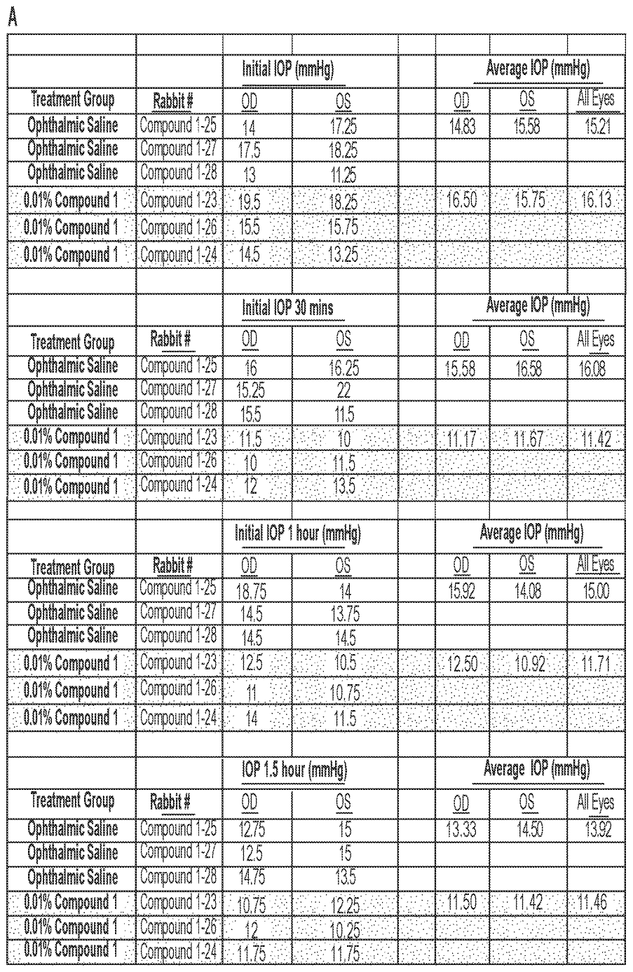

Overview of Experimental Layout (see, for example, FIG. 2)

### Animal Weight and Dosing:

| | | | R-DOI | | | |
|---|---|---|---|---|---|---|
| | | | HCl | | | |
| | | | (μg) | R-DOI | R-DOI | R-DOI |
| Animal | Weight | Treat- | dose/ | HCl | dose/ | dose |
| # | (kg) | ment | animal | μg/kg | animal | μg/kg |
| El | 2.82 | BSS; | 0 | 0.000 | 0 | 0 |
| | | 0.5% | | | | |
| | | CMC | | | | |
| E2 | 2.76 | BSS; | 0 | 0.000 | 0 | 0 |
| | | 0.5% | | | | |
| | | CMC | | | | |
| E3 | 2.88 | 0.05% | 50 | 17.36 | 44.90 | 15.59 |
| | | R-DOI; | | | | |
| | | BSS; | | | | |
| | | 0.5% | | | | |
| | | CMC | | | | |
| E4 | 2.70 | 0.05% | 50 | 18.52 | 44.90 | 16.63 |
| | | R-DOI; | | | | |
| | | BSS; | | | | |
| | | 0.5% | | | | |
| | | CMC | | | | |
| E5 | 3.06 | 0.01% | 10 | 3.268 | 8.980 | 2.935 |
| | | R-DOI; | | | | |
| | | BSS; | | | | |
| | | 0.5% | | | | |
| | | CMC | | | | |
| E6 | 2.82 | 0.01% | 10 | 3.546 | 8.980 | 3.184 |
| | | R-DOI; | | | | |
| | | BSS; | | | | |
| | | 0.5% | | | | |
| | | CMC | | | | |

Animals from the acute dosing were continued through the repeated dosing study. Animals were dosed at the indicated concentration 2 times on day 1 and then dosed 4 times per day for 3 subsequent days. Doses were administered at 50 μl per eye into both eyes of the animal.

### Outcomes:

Control Group (E1/E2; 0% drug): No observable changes; Animals were relaxed in cage grooming; All Normal Behaviors; No CNS, Ocular Behavioral or Systemic Changes. Response to Auditory Stimulation Normal.

Low Dose (E5/E6; 0.01% drug): No observable changes; Animals were relaxed in cage grooming; All Normal Behaviors; No CNS, Ocular Behavioral or Systemic Changes. Animals Eating and Grooming during observation. Response to Auditory Stimulation Normal.

Mid Dose (E3/E4; 0.05% drug): No observable changes; Animals were relaxed in cage grooming; All Normal Behaviors; No CNS, Ocular Behavioral or Systemic Changes. Animals Eating and Grooming during observation. Response to Auditory Stimulation Normal.

### Weight Changes Across 1 Week of Dosing:

| | | | | | | | 1 | 1 |
|---|---|---|---|---|---|---|---|---|
| | | 1 dose/day | 2 dose/day | 4 dose/day | 4 dose/day | 4 dose/day | week | week |
| Animal | Wcight | Treatment | Treatment | Treatment | Treatment | Treatment | weight | weight |
| # | (kg) | day #1 | day #2 | day #3 | day #4 | day #5 | (kg) | change |
| E1 | 2.82 | BSS | BSS | BSS | BSS | BSS | 2.88 | 0.06 |
| | | 0.5% | 0.5% | 0.5% | 0.5% | 0.5% | | |
| | | CMC | CMC | CMC | CMC | CMC | | |
| E2 | 2.76 | BSS | BSS | BSS | BSS | BSS | 2.89 | 0.13 |
| | | 0.5% | 0.5% | 0.5% | 0.5% | 0.5% | | |
| | | CMC | CMC | CMC | CMC | CMC | | |
| E3 | 2.88 | 0.1% | 0.05% | 0.05% | 0.05% | 0.05% | 2.96 | 0.08 |
| | | R-DOI; | R-DOI; | R-DOI; | R-DOI; | R-DOI; | | |
| | | 0.5% | 0.5% | 0.5% | 0.5% | 0.5% | | |
| | | CMC | CMC | CMC | CMC | CMC | | |
| E4 | 2.70 | 0.1% | 0.05% | 0.05% | 0.05% | 0.05% | 2.79 | 0.09 |
| | | R-DOI; | R-DOI; | R-DOI; | R-DOI; | R-DOI; | | |
| | | 0.5% | 0.5% | 0.5% | 0.5% | 0.5% | | |
| | | CMC | CMC | CMC | CMC | CMC | | |
| E5 | 3.06 | 0.01% | 0.01% | 0.01% | 0.01% | 0.01% | 3.17 | 0.11 |
| | | R-DOI; | R-DOI; | R-DOI; | R-DOI; | R-DOI; | | |
| | | 0.5% | 0.5% | 0.5% | 0.5% | 0.5% | | |
| | | CMC | CMC | CMC | CMC | CMC | | |
| E6 | 3.82 | 0.01% | 0.01% | 0.01% | 0.01% | 0.01% | 2.89 | 0.07 |
| | | R-DOI; | R-DOI; | R-DOI; | R-DOI; | R-DOI; | | |
| | | 0.5% | 0.5% | 0.5% | 0.5% | 0.5% | | |
| | | CMC | CMC | CMC | CMC | CMC | | |

Assessment: During the 2 hour observation windows, there were no adverse or abnormal events observed for rabbits treated with control, 0.01%, or 0.05% R-DOI HCl as indicated. Ophthalmic drops were tolerated with no signs or symptoms of irritability. Neither signs of dry eye nor excessive tear production was observed. There was no damage to corneal surfaces, barrier intactness, inflammation, or neovascularization. There was also no loss of weight with repeated dosing. Animals gained weight following 1 week of dosing and handling. There was no significant change in differences of weight gain within 1 week of initiation of dosing.

**If there was any behavioral observation made, it was anecdotal: Rabbit E3 had a personality from the beginning that was withdrawn. It was housed in the bottom enclosure with little light present from the overhead fluorescent illumination. It stayed near the back of the enclosure. At about the third day of treatment, the rabbit's behavior changed to interactive. It no longer cowered at the back of the cage when entering room or when attempting to acquire the rabbit. Its behavioral responses went from a depressed like state to a normal interactive rabbit. This was noted by both researchers in the room.

### Effects of R-DOI on Intraocular Pressure:

The IOP was assessed as a component of clinical outcomes for the animals in the studies described herein. The table below represents the effect of R-DOI on IOP 2 hours after dosing with the indicated concentration of R-DOI. Eyes were individually assessed and are indicated as IOP for the right (OD) or left (OS) eye.

| | | Avg | Avg | | | Avg | Ave | | | Avg | Avg |
|---|---|---|---|---|---|---|---|---|---|---|---|
| | | OD | OD | Delta | | OS | OS | Delta | | Delta | % |
| Animal | | Starting | 2 hpt | OD | % | Starting | 2 hpt | OS | % | Eye | change |
| # | Treatment | IOP | IOP | (2 h) | Change | IOP | IOP | (2 h) | Change | Group | Group |
| E1 | BSS | 11.50 | 12.50 | 1.00 | 8.70 | 14.25 | 14.25 | 0.00 | 0.00 | | |
| | 0.55 | | | | | | | | | | |
| | CMC | | | | | | | | | | |
| E2 | BSS | 13.25 | 13.50 | 0.25 | 1.89 | 10.50 | 15.25 | 4.75 | 45.24 | 1.50 | 5.29 |
| | 0.5% | | | | | | | | | | |
| | CMC | | | | | | | | | | |
| E3 | 0.1% | 19.00 | 11.50 | −7.50 | −39.47 | 15.00 | 13.25 | −1.75 | −11.67 | | |
| | R-DOI; | | | | | | | | | | |
| | 0.5% | | | | | | | | | | |
| | CMC | | | | | | | | | | |
| E4 | 0.1% | 17 | 13.25 | −3.75 | −22.06 | 15.75 | 9.00 | −6.75 | −42.86 | −4.94 | −30.77 |
| | R-DOI; | | | | | | | | | | |
| | 0.5% | | | | | | | | | | |
| | CMC | | | | | | | | | | |
| E5 | 0.01% | 15.00 | 12.25 | −8.50 | −56.67 | 20.75 | 13.50 | −7.25 | −34.94 | | |
| | R-DOI; | | | | | | | | | | |
| | 0.5% | | | | | | | | | | |
| | CMC | | | | | | | | | | |
| E6 | 0.01% | 14 | 7.00 | −7.00 | −50.00 | 15.25 | 7.00 | −8.25 | −54.10 | −7.75 | −53.33 |
| | R-DOI; | | | | | | | | | | |
| | 0.5% | | | | | | | | | | |
| | CMC | | | | | | | | | | |

Assessment: Intraocular pressure in rabbits is highly dependent on time of day measurements due to circadian rhythm regulation of aqueous humor flow. As can be seen for control animals, IOP increased over the 2 hour treatment/assessment window for yes assessed. In contrast, eyes given R-DOI as a single dose of 0.1% or 0.01% had decreased IOP with the greatest reduction of IOP observed for the lower R-DOI dosages.

### Example 3

Non-Limiting, Exemplary Findings Discussed within:
The NCE Compound 1 was formulated within an ophthalmic eye drop with BSS and 0.5% carboxymethylcellulose (CMC) at percentages of 0.1% (5 mg/5 ml) and lower with a pH of 7.0. Solubility of the compound was good with no remaining precipitates. This solution was diluted 1:10 in ophthalmic BSS 0.5% CMC for the present studies.
0.01% Compound 1 in an acute single dosing did not exhibit any acute ocular toxicity, irritability or adverse ocular reactions.
No readily apparent adverse events, including ocular, systemic, behavioral or otherwise were noted for the 0.01% Compound 1 dose administered.
Administration of Compound 1 decreased intraocular pressure with a single dose.
The effects of Compound 1 on decreasing intraocular pressure were sustained for a period of at least 3 days post administration.
Administration of Compound 1 increased tear production as measured by Schirmer's test.
The effects of Compound 1 on increased tear production were sustained for a period of at least 7 days post administration.
Return to Baseline IOP took approximately 12 days post single dose administration of 0.01% Compound 1.

### Preparation of Ophthalmic Solution of Compound 1 for Animal Ocular Studies:

### Calculations and Conversions of Compound 1, as Well as Dilutions of Stock Solution:

| Compound 1 | | MW 237.33 | | | Dilution |
|---|---|---|---|---|---|
| Weight/volume | % conc. | Molar Conc. | | Solution | Solution |
| 100 mg/100 mL | 0.10% | 4.21 | mM | Stock Solution | Ophthalmic |
| | | | | Made at 5 | BSS with 0.5% |
| | | | | mg/5 mL | CMC |
| 10 mg/100 mL | 0.01% | 421.35 | μM | 1:10 Dilution of | Ophthalmic |

| | |
|---|---|
| 0.1% Stock | BSS with 0.5% |
| | CMC |

### Formulation of the NCE, Compound 1 Ophthalmic Eye Drops:

A 5 mL Stock of 0.1% of Compound 1 in Ophthalmic BSS with 0.5% Carboxymethylcellulose (CMC) was made with 5 mg of Compound 1 pH was at 7.0.

Herein we determine if the ocular composition of 0.01% Compound 1 with 0.5% carboxymethylcellulose in ophthalmic BSS:

Produced any observable behavioral effects.

Was tolerated by the eye without adverse ocular effects, including absence of:
Apparent Ocular Irritability
Induction of Inflammation
Dry Eye or Excessive Tearing
Induction of Scleral Reddening
Reduced IOP
Maintained Reduction in IOP
Increased Tear Film Production
Maintained Increased Tear Film Production

### Overview of Experimental Layout:

Rabbits were assessed for clinically clear eyes by slit lamp ophthalmic scoring. All eyes of all rabbits were normal.

Each eye of each rabbit was then assessed for initial IOP by tonometer.

Each eye of each rabbit was assessed for tear film production by Schirmer's Tear Flo Strips at a 30 second interval of analysis. Tear measures were evaluated by a digital caliper to determine mm of tears collected.

Rabbits were sorted into two groups attempting to balance IOP numbers (one higher, one lower animal per group).

Following balancing animals were treated with 50 μL of Control ophthalmic BSS/0.5% CMC or 0.01% Compound 1 in ophthalmic BSS/0.5% CMC.

Any behavioral changes were continuously monitored over the next 2.5 hours (FIG. 3).

IOP determinations were made at 30 minutes, 1 hour, 1.5 hours and 2.5 hours-eyes were examined closely for any adverse effects during these IOP measures.

3 days post treatment at the same time initial measures were made, IOP and tear film production were re-assessed to examine if effects were sustained post 1 treatment.

### Animal Weight and Dosing:

| | | | | Compound | |
|---|---|---|---|---|---|
| | Weight | Treatment | Volume | 1 Dose | Compound |
| Rabbit # | (kg) | Group | Administered | (μg)/animal | 1 μg/kg |
| Compound | 4.36 | BSS; 0.5% | 50 μL/Eye | 0 | 0.000 |
| 1-25 | | CMC | | | |
| Compound- | 4.72 | BSS; 0.5% | 50 μL/Eye | 0 | 0.000 |
| 27 | | CMC | | | |
| Compound | 4.54 | BSS; 0.5% | 50 μL/Eye | 0 | 0.000 |
| 1-28 | | CMC | | | |
| Compound | 3.82 | 0.01% | 50 μL/Eye | 10 | 2.618 |
| 1-23 | | Compound 1 | | | |
| Compound | 4.58 | 0.01% | 50 μL/Eye | 10 | 2.183 |
| 1-26 | | Compound 1 | | | |
| Compound | 4.26 | 0.01% | 50 μL/Eye | 10 | 2.347 |
| 1-24 | | Compound 1 | | | |

### Behavioral and Ocular Irritability Outcomes:

Control Group (Compound 1-25, -27, -28; 0% drug in BSS; 0.5% CMC): No observable changes; Animals were relaxed in cage grooming; All Normal Behaviors; No CNS, Ocular, Behavioral, or Systemic Changes. Response to Auditory Stimulation was Normal.

Treatment Arm (Compound 1-23, -24, -26; 0.01% Compound 1 drug in BSS; 0.5% CMC): No observable changes; Animals were relaxed in cage grooming; All Normal Behaviors; No CNS, Ocular, Behavioral, or Systemic Changes. Animals were eating and grooming during observation. Response to Auditory Stimulation was Normal.

Assessment: During the 2.5 hour observation window (as well as in the ensuing days post treatment), there were no adverse or abnormal events observed for rabbits treated with control or 0.01% Compound 1 as indicated. Animals did not display any signs of ocular irritability following dosing. There were no observed blinking, ocular redness, excessive epiphora, dry eye, swelling or irritant responses. There were no readily observed systemic effects of dosing (i.e. Increased respiration or heartrate). 0.01% Compound 1 dosing in each eye appeared to be acutely safe systemically, behaviorally and ocularly.

### Effects of Compound 1 on Intraocular Pressure:

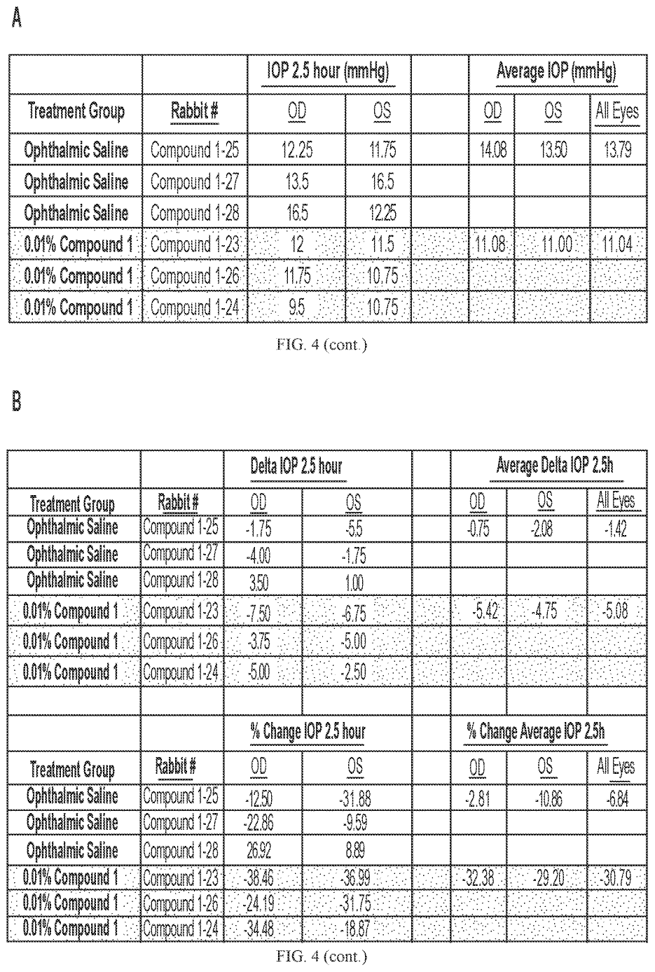

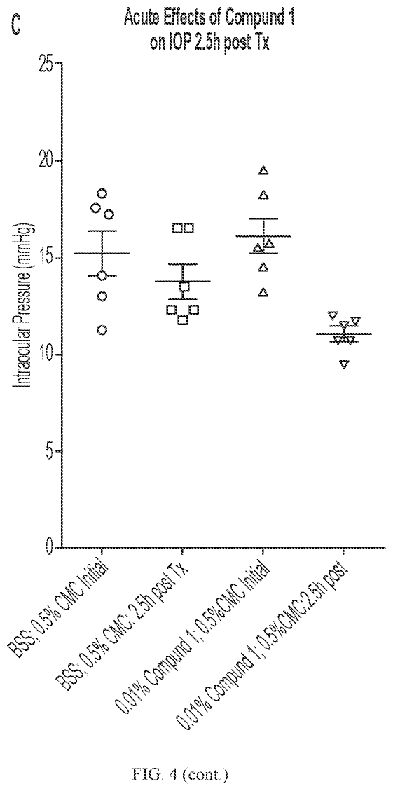

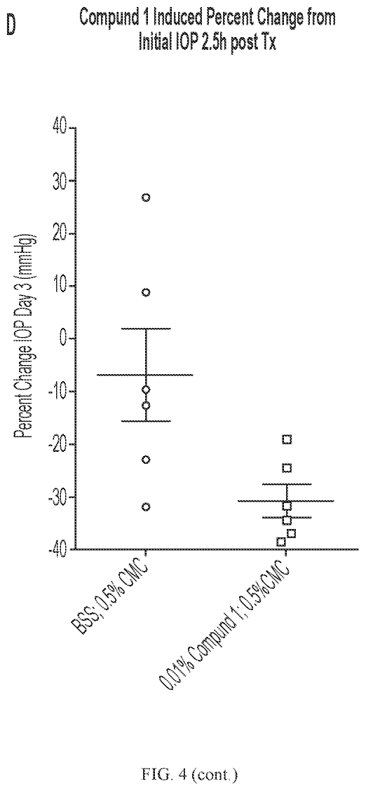

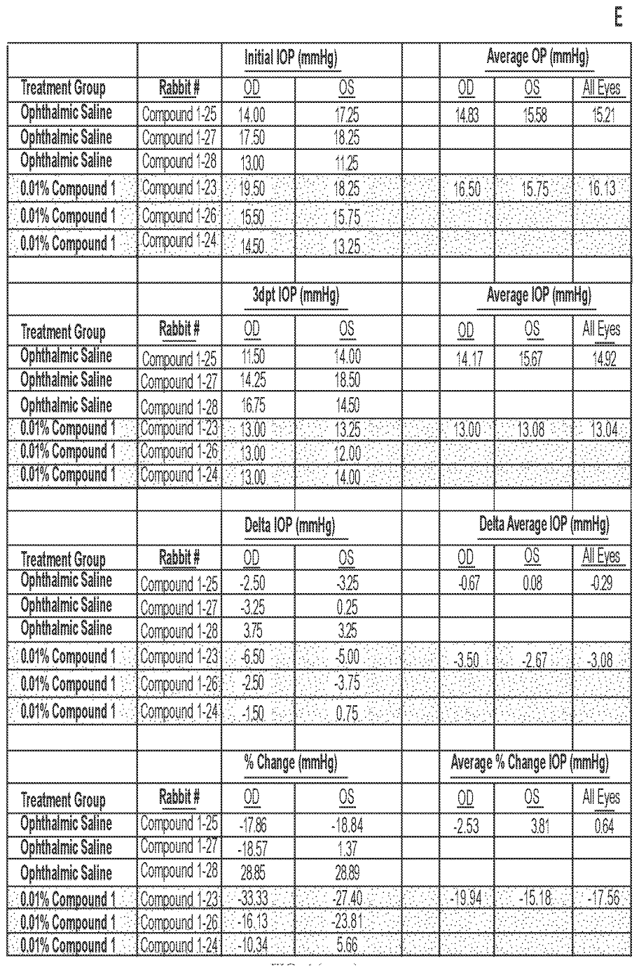

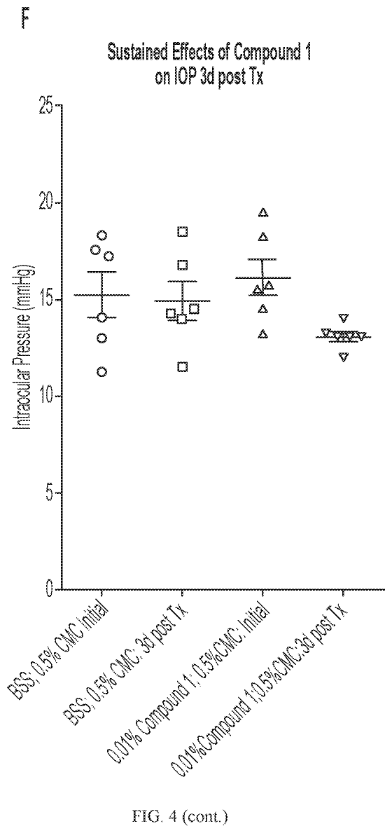

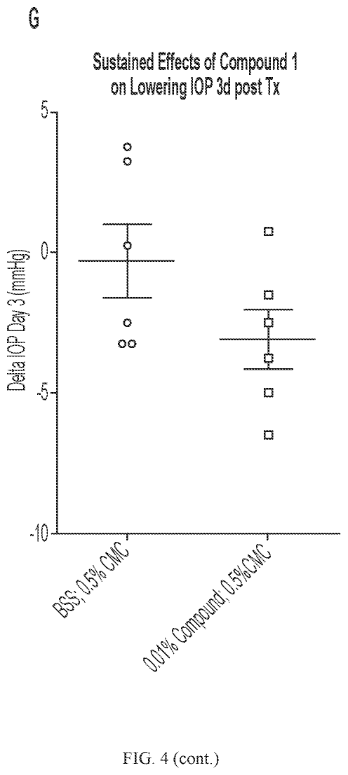

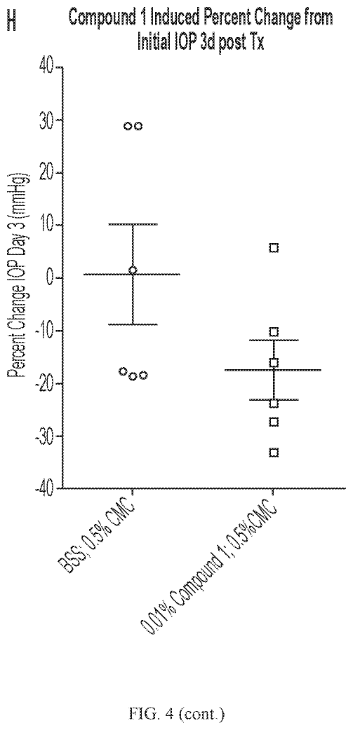

The IOP was assessed via a TonoVet rebound Tonometer. The tables herein (see, for example, FIG. 4) represent the effect of Compound 1 on IOP at time intervals within the first 2.5 hours after dosing with 0.01% Compound 1 in BSS with 0.5% CMC. Eyes were individually assessed and are indicated as IOP for the right (OD) or left (OS) eye. 3 days post treatment eyes were reassessed for IOP changes to see if the effects of treatment lasted over a prolonged period.

Assessments: Intraocular pressure in rabbits is dependent on time of day measurements due to circadian rhythm regulation of aqueous humor flow. As can be seen for control animals, IOP initially increased during the 2.5 hour treatment/assessment window, with an eventual decrease in IOP of 1.42 across the observation window. Other studies have observed this identical waxing and waning of IOP during a day with peaks and troughs identified where control BSS treatments decrease IOP by approximately 1.2 mm Hg (similar to that observed here). In contrast, eyes given 0.01% Compound 1 as a single dose had decreased IOP at all points across the 2.5 hour observation window.

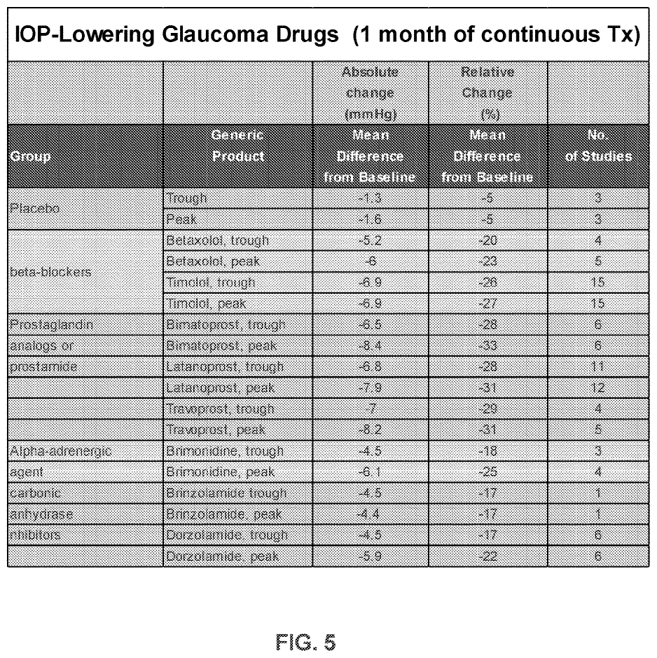

An analysis of the effects of current anti-glaucoma drugs are shown herein (see, for example, FIG. 5). This meta-analysis indicates one aspect we can be striving for in development of an IOP-lowering anti-glaucoma medication. Also, included is a sorting by their basic mechanisms of action. One thing to note from a side effect perspective-given these drugs MOAs, many of them change tear production, or ocular fluid levels/flow in order to reduce IOP. The consequence of that is that long term treatment results in dry eye symptoms from lack of tear production. Please note later data on tear film in eyes treated with Compound 1. That can help differentiate its potential versus current IOP-lowering glaucoma drugs. In addition, these drugs eventually fail, as the optic nerve eventually goes through apoptosis and death despite IOP lowering effects. Without wishing to be bound by theory, 5-HT2 agonists can also have neuroprotective effects and can be an addition to the full disease pathology associated with glaucoma. Whether Compound 1 also has these neuroprotective effects against apoptosis will be assessed.

Analysis of the previous data herein, indicates that Compound 1 has similar % difference IOP lowering effects compared to current therapeutics as provided in a single dose, and that single dose maintains efficacy in a non-diseased eye model for a period of at least 3 days. These preliminary results are promising for its use as a therapeutic, depending on its effects on IOP following long-term administration.

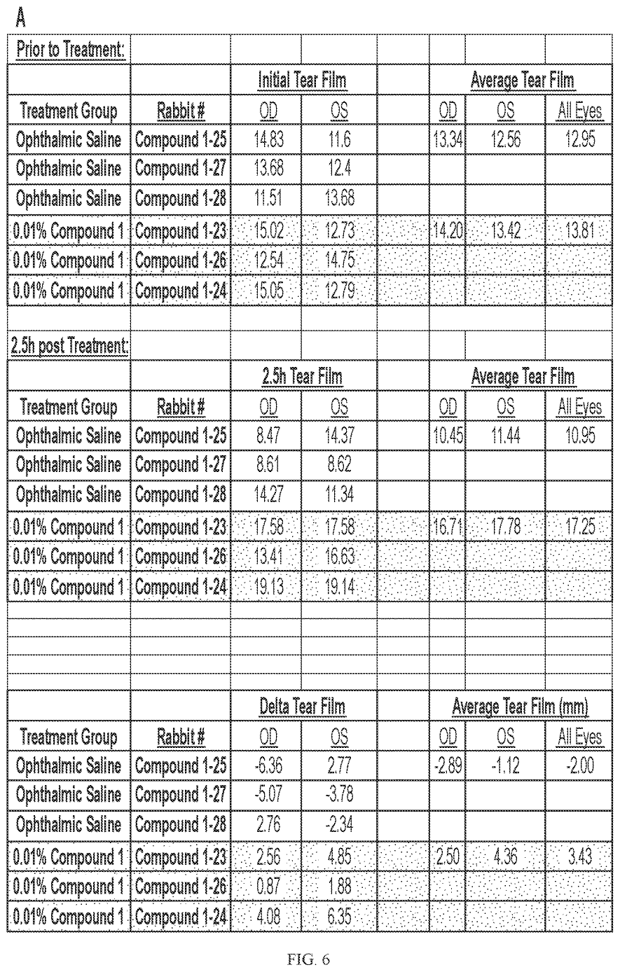

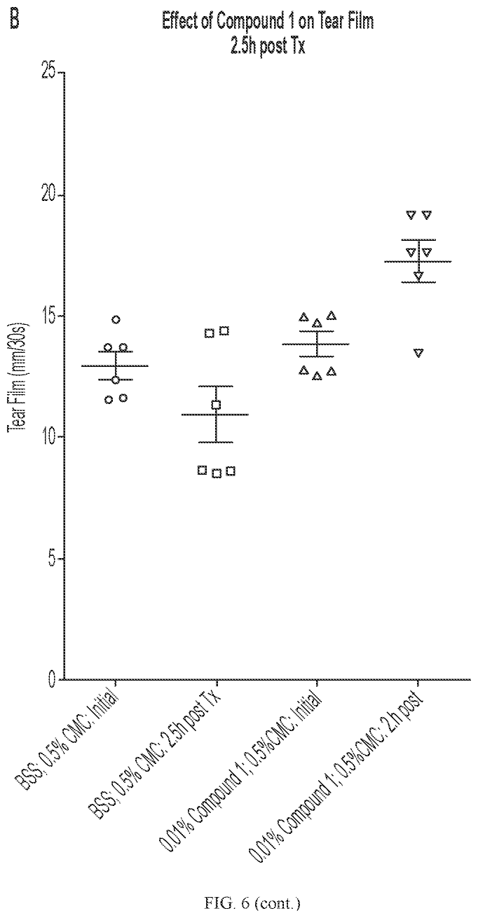

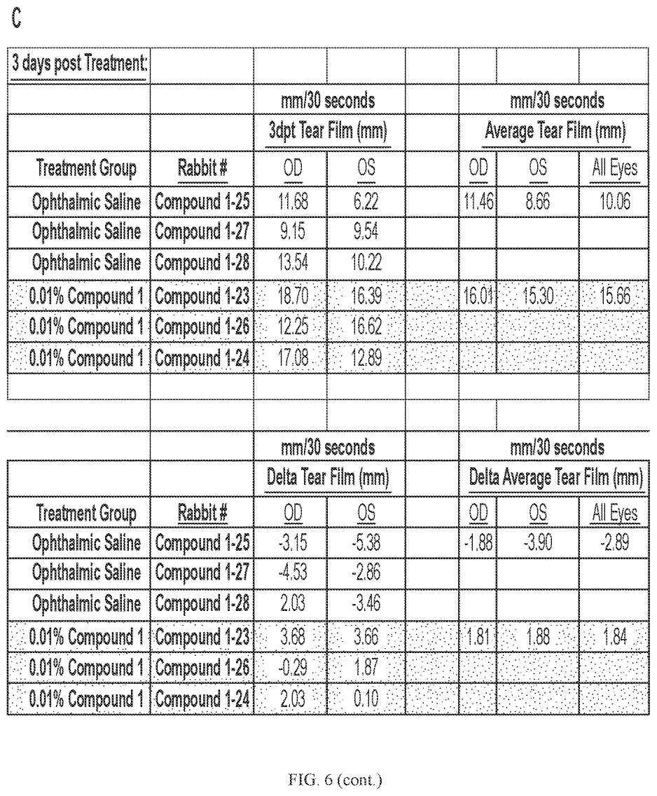

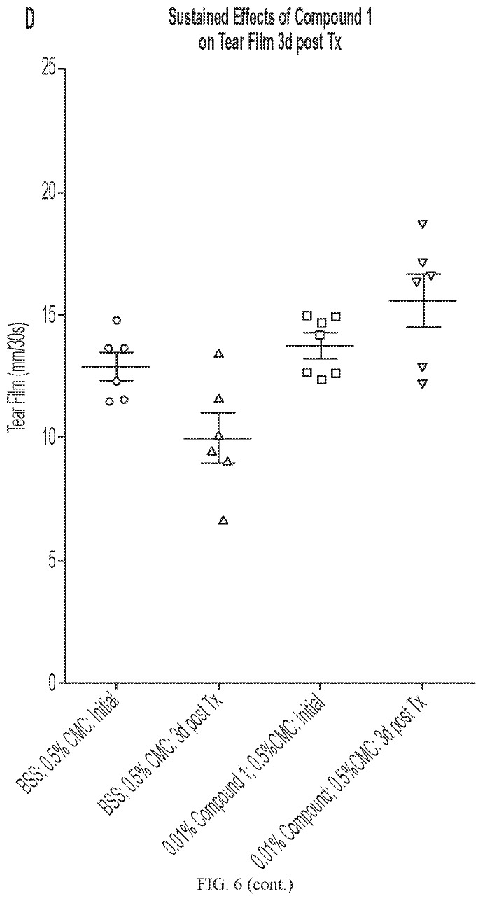

Effects of Compound 1 on Tear Production: Current IOP lowering glaucoma drugs eventually have effects on tear production resulting in dry eye conditions. Further, there are additional associations between dry eye and glaucoma, with co-incidence of the two conditions quite common. Ocular serotonin levels have been implicated with Dry Eye, indicating that 5-HT receptors can play a role in dry eye development and can modulate those processes. Selective 5-HT agonists/antagonists can reverse symptoms of dry eye-a very large ophthalmic market. The effects of Compound 1 on tear film production (see, for example, FIG. 6 panels A-D) were therefore assessed by a Schirmer's Tear Flo strip and measured using a digital micrometer caliper.

Control eyes treated with ophthalmic BSS/0.5% CMC and subsequently repeatedly assessed for IOP exhibited a marked decline in tear film at 2.5 h post treatment. In contrast, eyes treated with 0.01% Compound 1 in ophthalmic BSS 0.5% CMC exhibited a significant increase in tear production as assessed at 2.5 h post treatment (see, for example, FIG. 6 panel B). Whereas control eyes had a 2 mm reduction in tear film production, Compound 1 treated eyes had an average 3.4 mm increase in tear film production.

### Sustained Effects of Compound 1 on Tear Production:

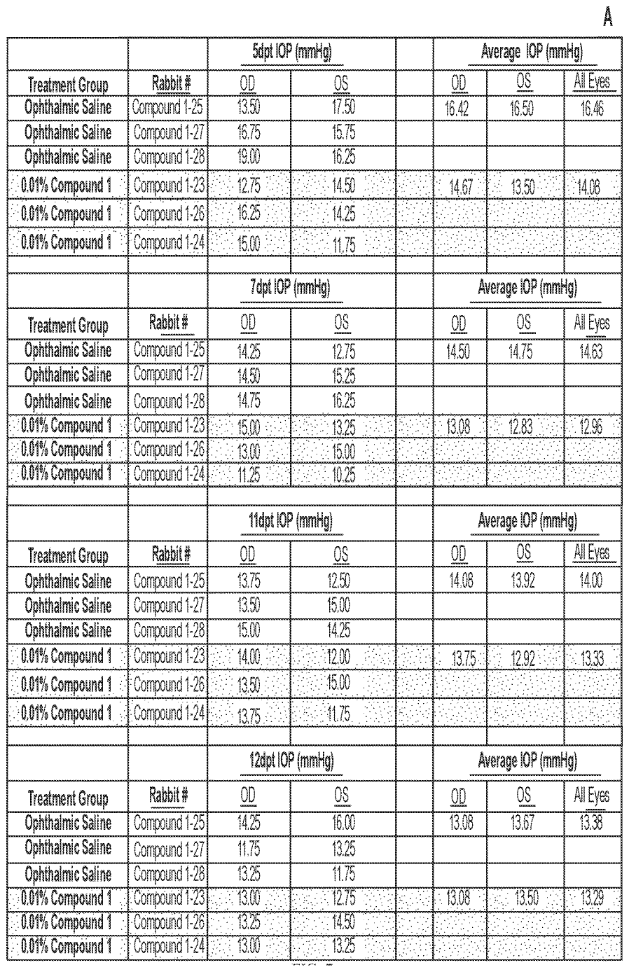

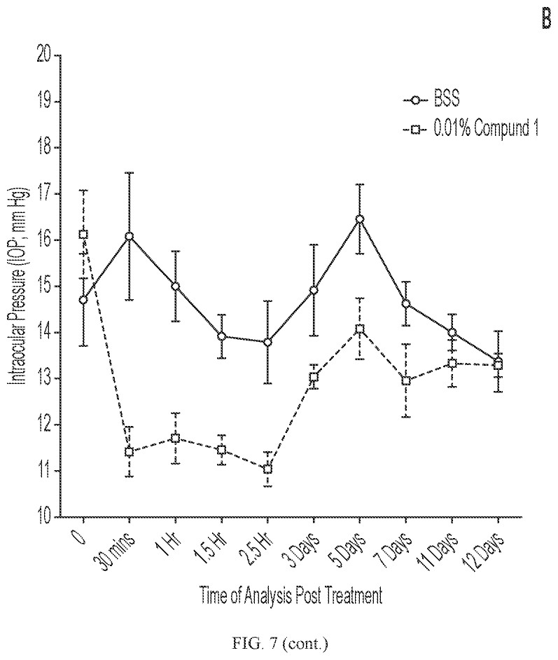

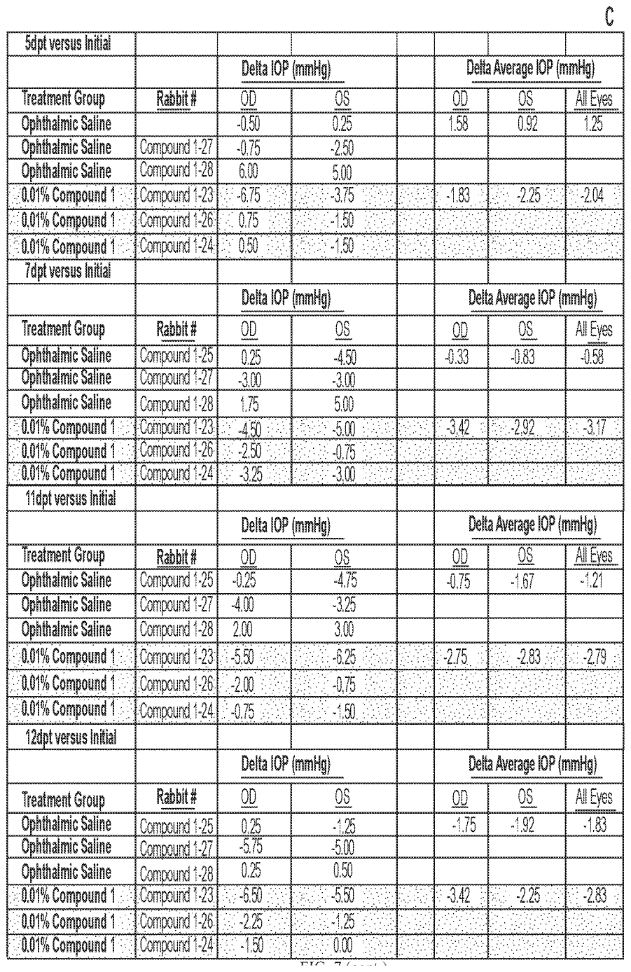

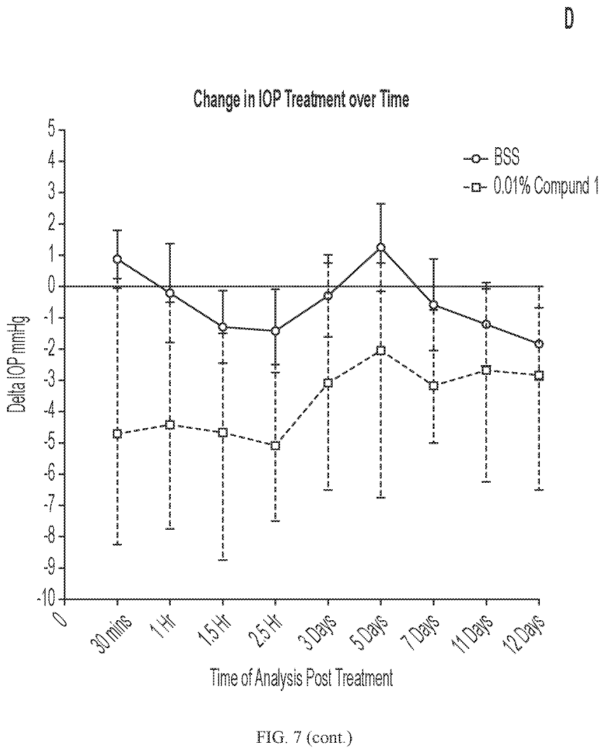

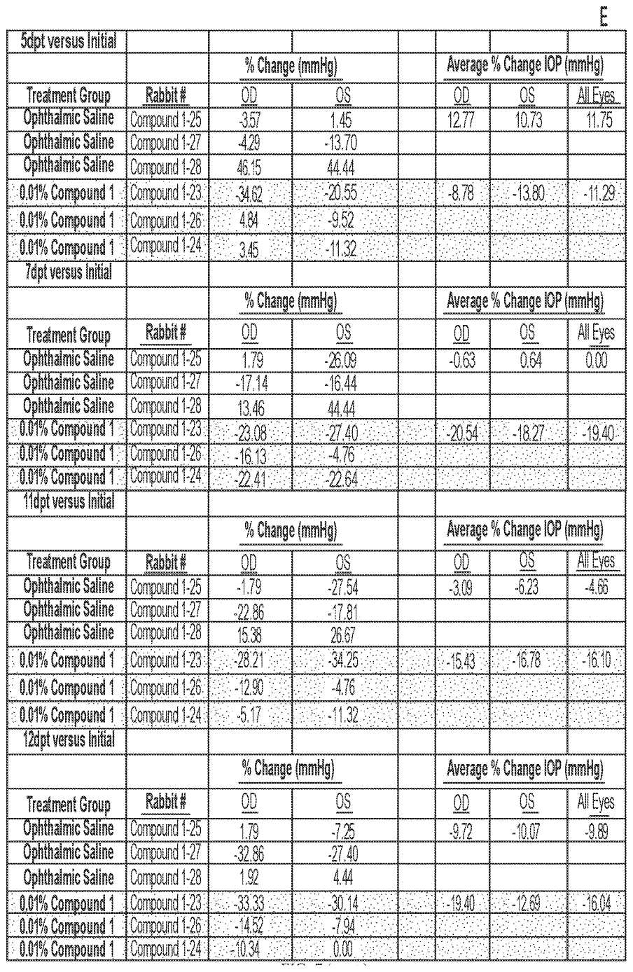

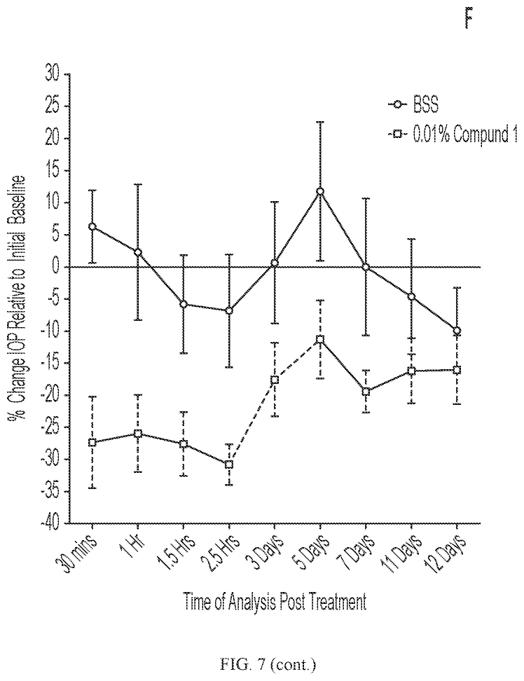

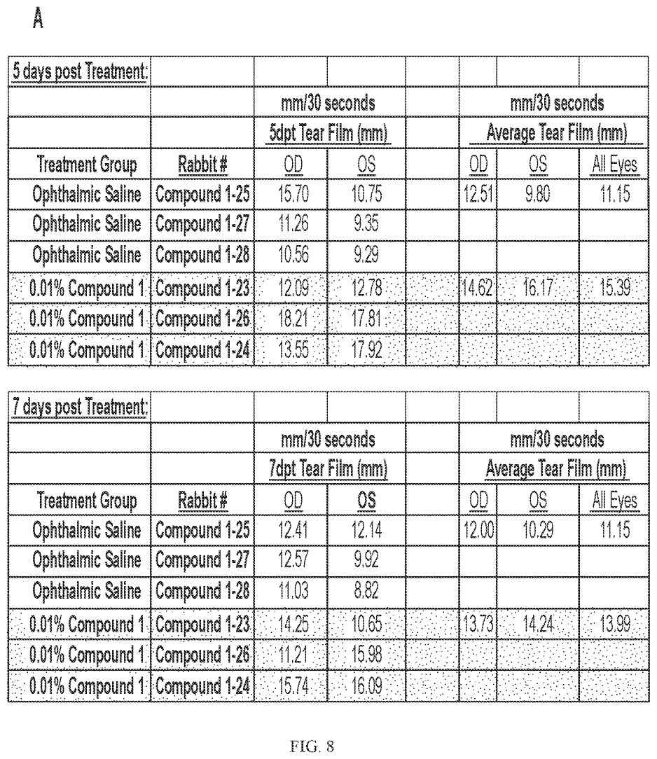

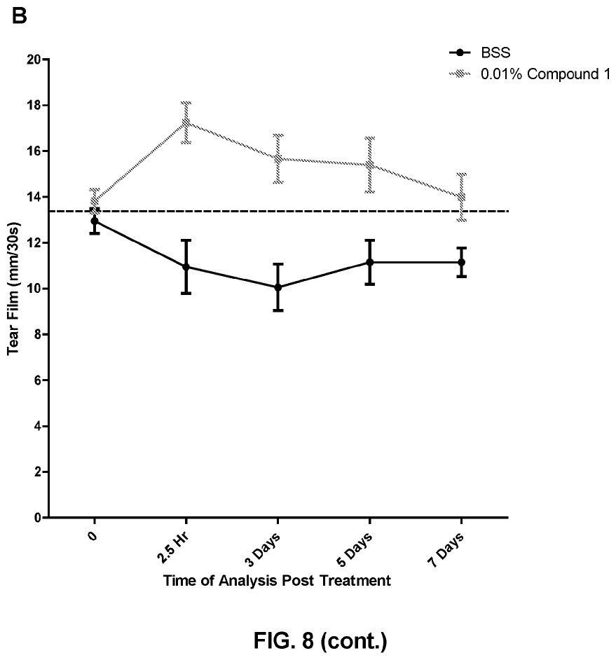

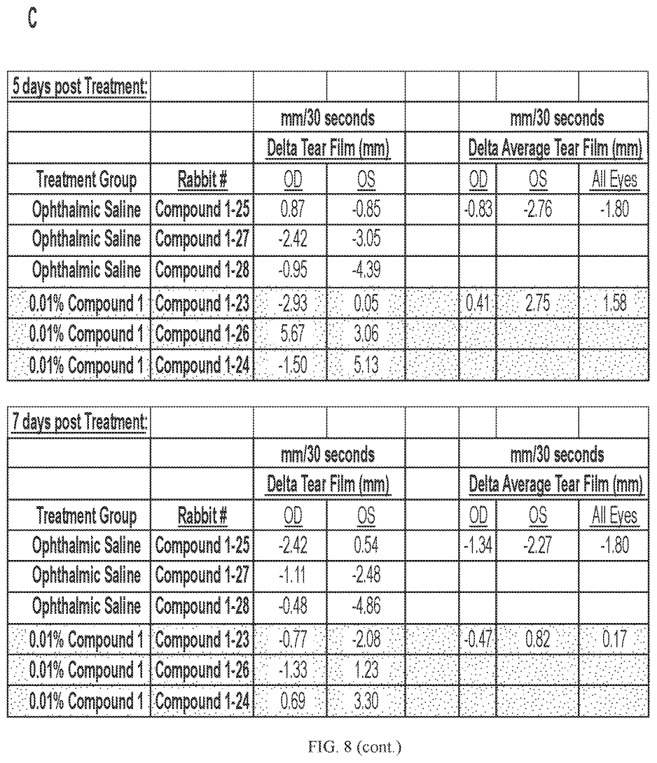

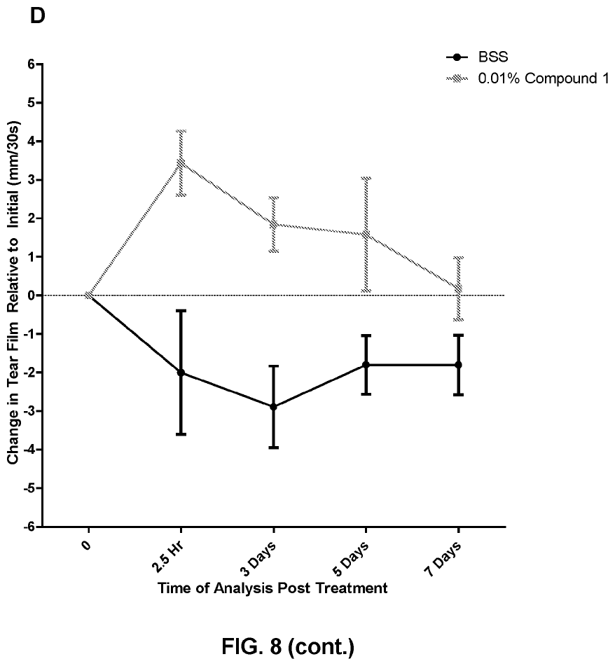

These increased tear film levels were sustained in Compound 1 treated eyes for at least 3 days post treatment (see, for example, FIGS. 6-8). Control eyes treated with ophthalmic BSS, exhibited an average reduction in tear film of approximately 2.9 mm. In contrast, eyes treated with Compound 1 had an average increase of tear film of approximately 1.9 mm.

Taken together, these results illustrate that Compound 1 has both IOP lowering and tear film increasing properties in an acute non-disease model. These combined activities do not exist in current therapeutics. These effects are promising for ocular indications. We will continue the short-term assessments of these eyes for both IOP and tear production to determine if these effects are lasting beyond the 3 day current assessments.

### Example 4

### Non-Limiting, Exemplary Findings Discussed Herein:

The R-DOI HCl was formulated within an ophthalmic eye drop with BSS and 0.5% carboxymethylcellulose (CMC) at percentages of 0.1% (5 mg/5 mL) and lower with a pH of 7.0. Solubility of the compound was good with no remaining precipitates. This solution was diluted 1:10 in ophthalmic BSS 0.5% CMC for the present studies to a final concentration of 0.01%.
0.01% R-DOI in an acute single dosing did not exhibit any acute ocular toxicity, irritability, or adverse ocular reactions.
As shown previously, no apparent adverse events, including ocular, systemic, behavioral, or otherwise were noted for the 0.01% R-DOI dose administered.
Administration of R-DOI acutely decreased intraocular pressure with a single dose that showed a significant decrease in IOP by 30 minutes post administration.
The effects of R-DOI on decreasing intraocular pressure were acute in nature and not sustained following 3 hours post treatment. By 5 hours post treatment, the R-DOI and control treated animals IOP closely matched and followed equivalent trends.
Administration of R-DOI increased tear production relative to initial and control treatment as measured by Schirmer's test.

Preparation of Ophthalmic Solution of R-DOI for animal ocular studies: An ophthalmic solution of 0.01% R-DOI HCl was formulated and delivered as described in the previous September Eleusis report. Treatments were delivered at 5 μg/eye or 10 μg/animal for R-DOI HCl, or approximately 4.5 μg/eye or 9 μg/animal for just the R-DOI component of the drug.

### Formulation of the NCE, R-DOI Ophthalmic Eye Drops:

A 5 mL Stock of 0.1% of Compound 1 in Ophthalmic BSS with 0.5% Carboxymethylcellulose (CMC) was made with 5 mg of R-DOI HCl, pH was at 7.0.

We can determine if a single dose of the ocular composition of 0.01% R-DOI with 0.5% carboxymethylcellulose in ophthalmic BSS:
1. Reproduced previous findings that R-DOI had IOP depressing effects.
2. Produced any readily observable behavioral effects.
3. Was tolerated by the eye without adverse ocular effects, including absence of:
a. Apparent Ocular Irritability
b. Induction of Inflammation
c. Dry Eye or Excessive Tearing
d. Induction of Scleral Reddening
4. Reduced IOP
5. Maintained Reduction in IOP
6. Increased Tear Film Production

### Overview of Experimental Layout:

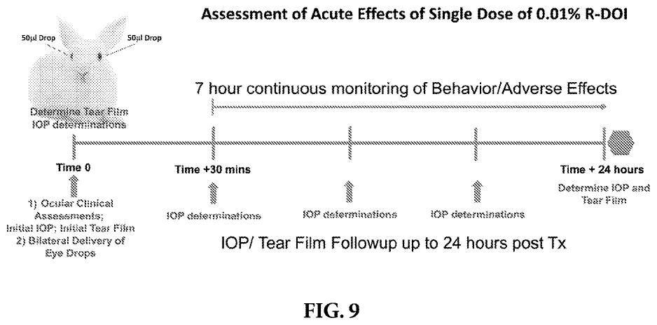

Rabbits were assessed for clinically clear eyes by slit lamp ophthalmic scoring. All eyes of all rabbits were normal.
Each eye of each rabbit was then assessed for initial IOP by tonometer.
Each eye of each rabbit was assessed for tear film production by Schirmer's Tear Flo Strips at a 30 second interval of analysis. Tear measures were evaluated by a digital caliper to determine mm of tears collected.
Rabbits were sorted into two groups attempting to balance IOP numbers (one higher, one lower animal per group).
Following animals were treated with 50 μL of Control ophthalmic BSS/0.5% CMC or 0.01% R-DOI HCl in ophthalmic BSS/0.5% CMC.
Any apparent behavioral changes were continuously monitored over the next 2.5 hours.
IOP determinations were made at 30 minutes, 1 hour, 2 hours and 3 hours, 5 hours, 7 hours and 24 hours-eyes were examined closely for any adverse effects during these IOP measures.
7 hours post treatment when IOP returned to control levels tear film production was assessed (see, for example, FIG. 9).

### Effects of Compound 1 on Intraocular Pressure:

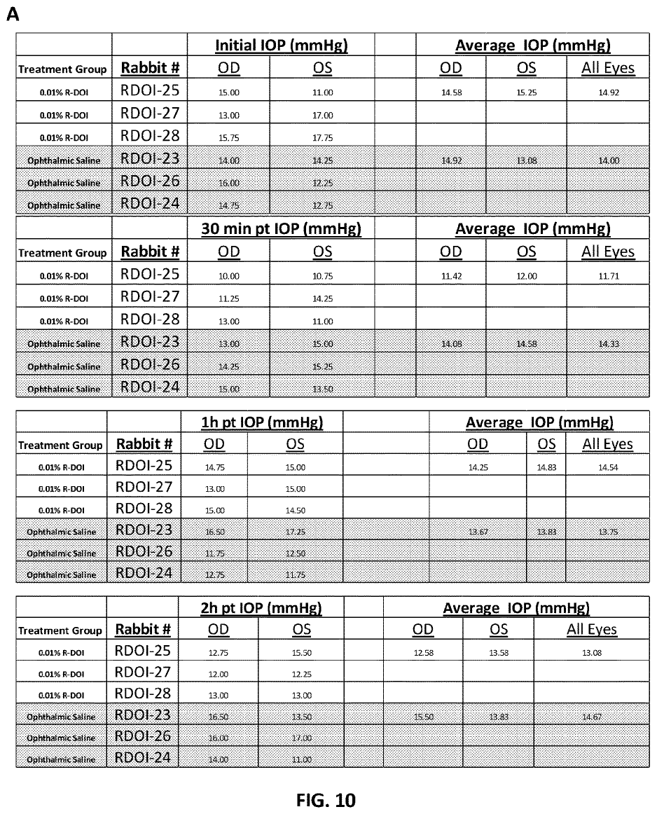

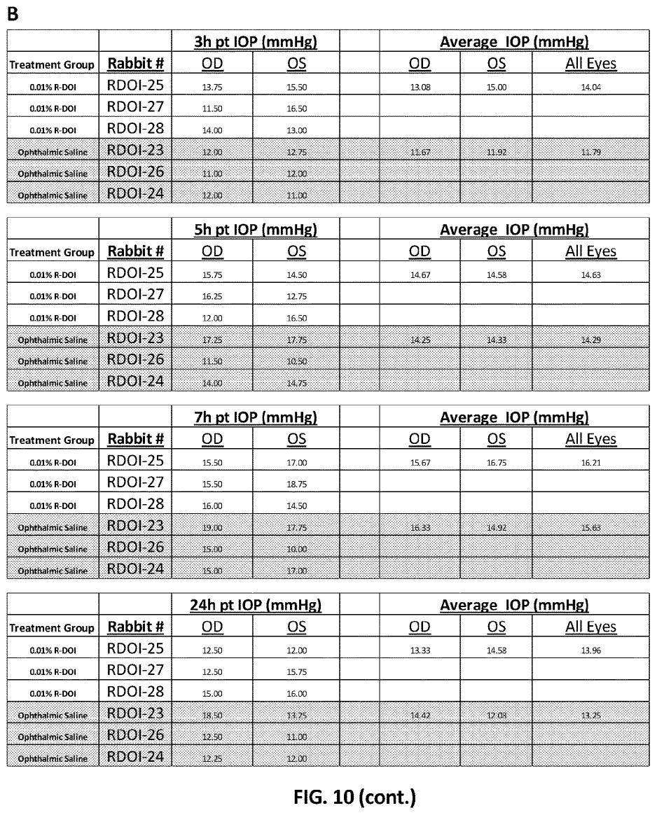

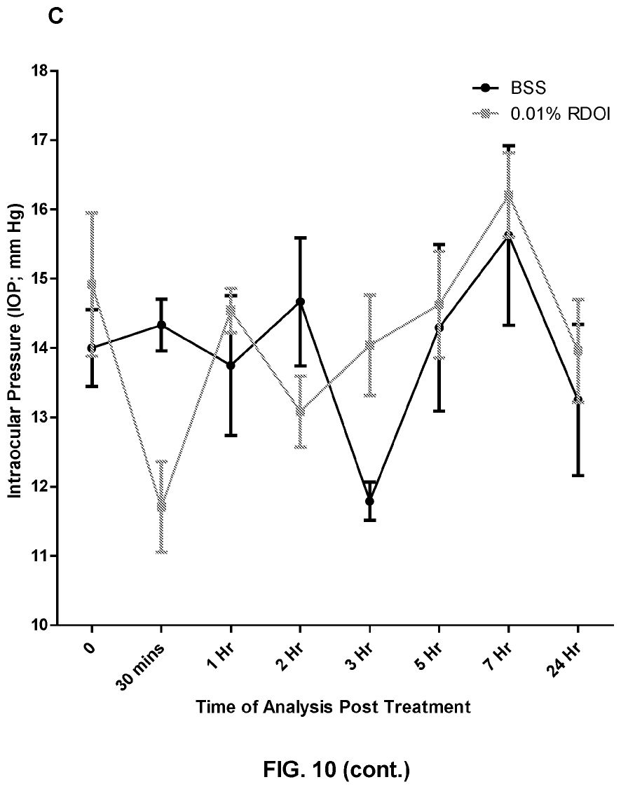

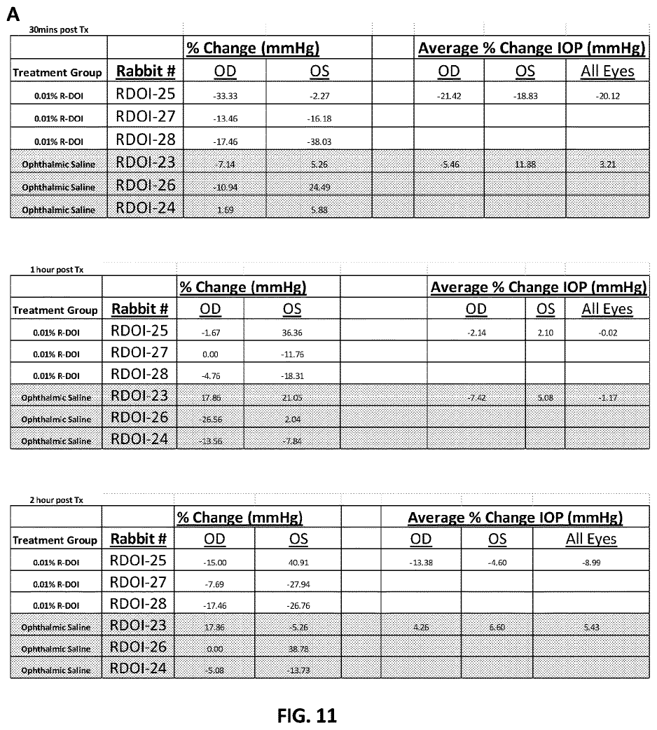

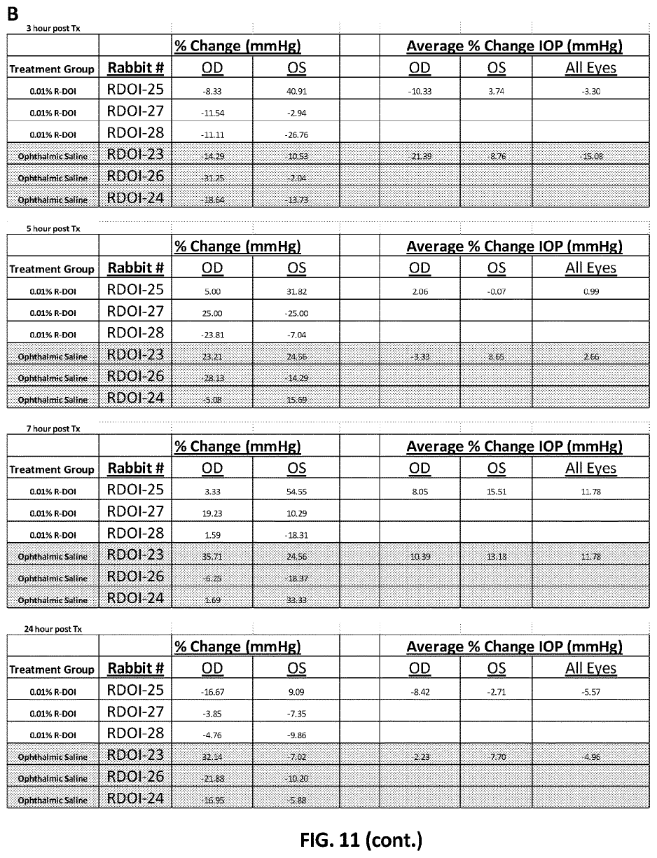

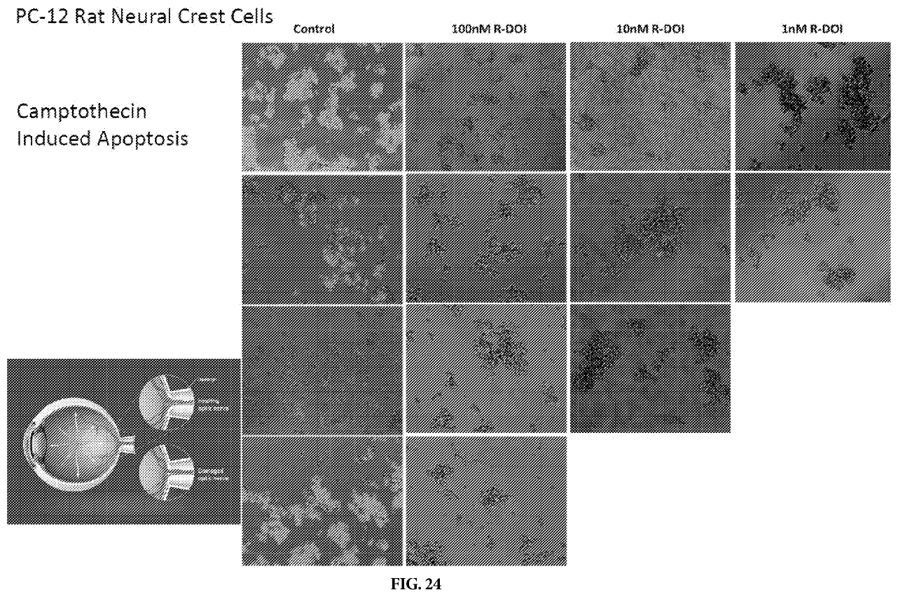

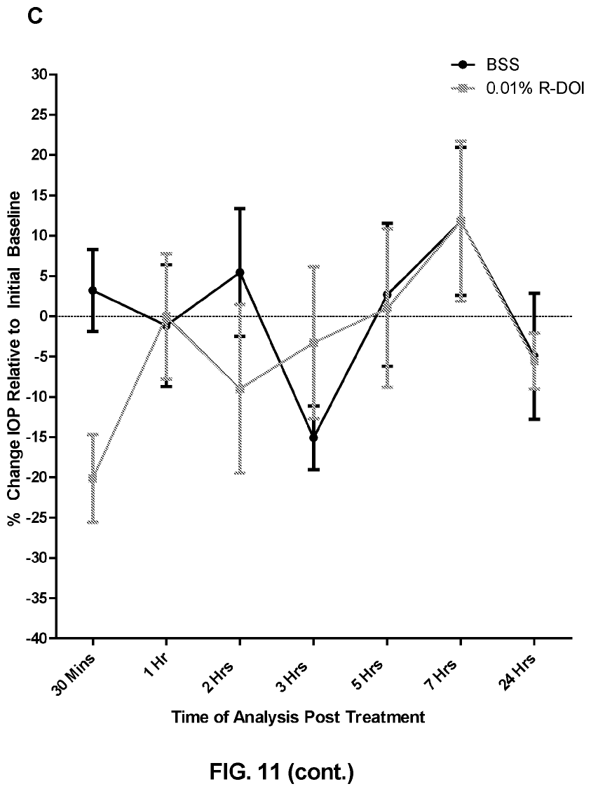

The IOP was assessed via a TonoVet rebound Tonometer. The tables herein (see, for example, FIGS. 10-11) represents the effect of R-DOI on IOP at the defined time intervals Eyes were individually assessed and are indicated as IOP for the right (OD) or left (OS) eye.

Assessments: Intraocular pressure in rabbits is highly dependent on time of day measurements due to circadian rhythm regulation of aqueous humor flow. As can be seen for control animals, IOP levels fluctuated during the 7 hour treatment/assessment window. Other studies have observed this identical waxing and waning of IOP during a day with peaks and troughs identified.

Effects of R-DOI on Tear Production: Current IOP lowering glaucoma drugs eventually have effects on tear production resulting in dry eye conditions. Further, there are additional associations between dry eye and glaucoma, with co-incidence of the two conditions common. Ocular serotonin levels have been correlated with Dry Eye, indicating that 5-HT receptors can play a role in dry eye development and, without wishing to be bound by theory, modulating those processes. Selective 5-HT agonists/antagonists can reverse symptoms of dry eye-a very large ophthalmic market. The effects of R-DOI on tear film production were therefore assessed by a Schirmer's Tear Flo strip and measured using a digital micrometer caliper.

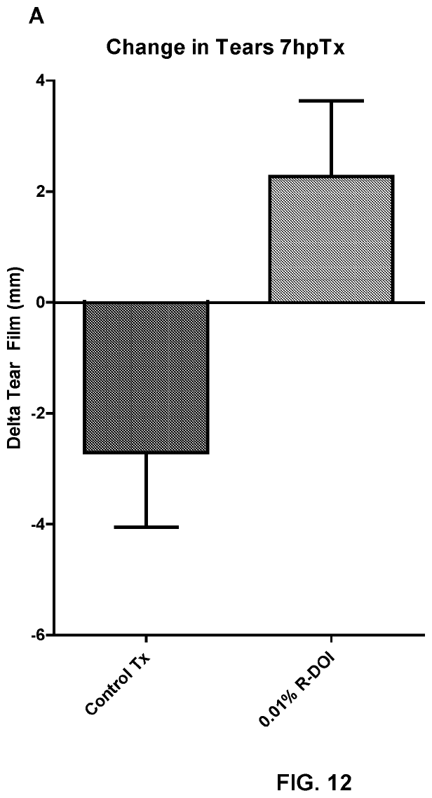

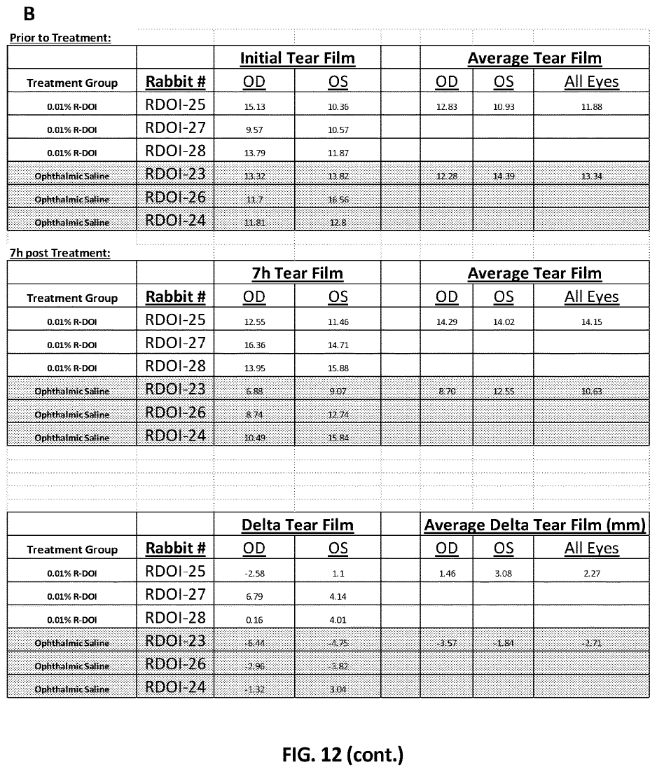

Control eyes treated with ophthalmic BSS/0.5% CMC and subsequently repeatedly assessed for IOP exhibited a marked decline in tear film at 7 h post treatment. In contrast, eyes treated with 0.01% R-DOI in ophthalmic BSS 0.5% CMC exhibited a significant increase in tear production as assessed at 7 h post treatment. Whereas control eyes had a 2.71 mm reduction in tear film production, R-DOI treated eyes had an average 2.27 mm increase in tear film production (see, for example, FIG. 12).

### Example 5

### Ocular Indications: Uveitis

Human Disease
Veterinary Equine Disease
   Inter-Relatedness of Findings from Other Studies

### Topical Ocular Safety

Topical Ocular Behavioral Effects
Topical Ocular Irritation and Tolerability
Direct Cellular Toxicity

### Dosing Regimen/Concentration

Information from in vivo assays
Information from in vitro assays
5-HT Receptor Agonist Outcomes and Importance of Selectivity

### Efficacy in Ocular Models

Tear Production
Intraocular Pressure
Herpetic Eye Disease

### Mechanisms of Action

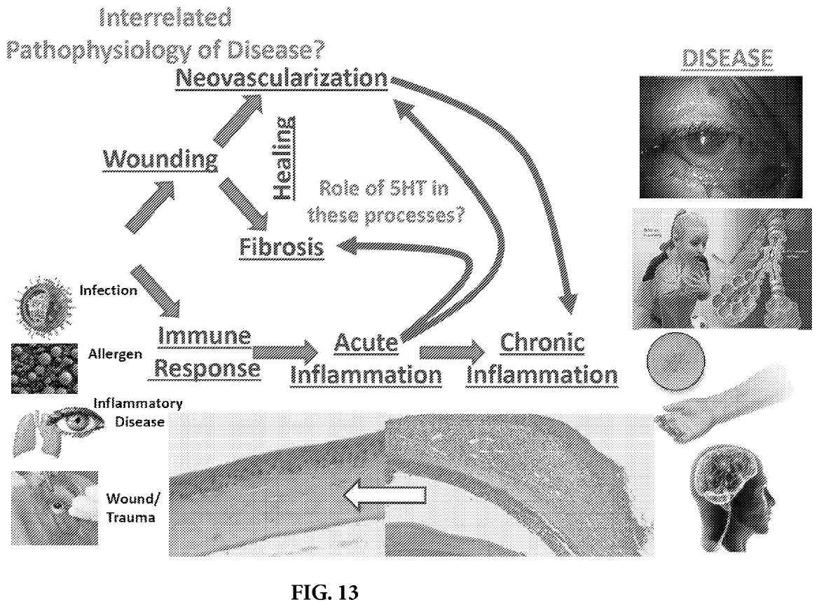

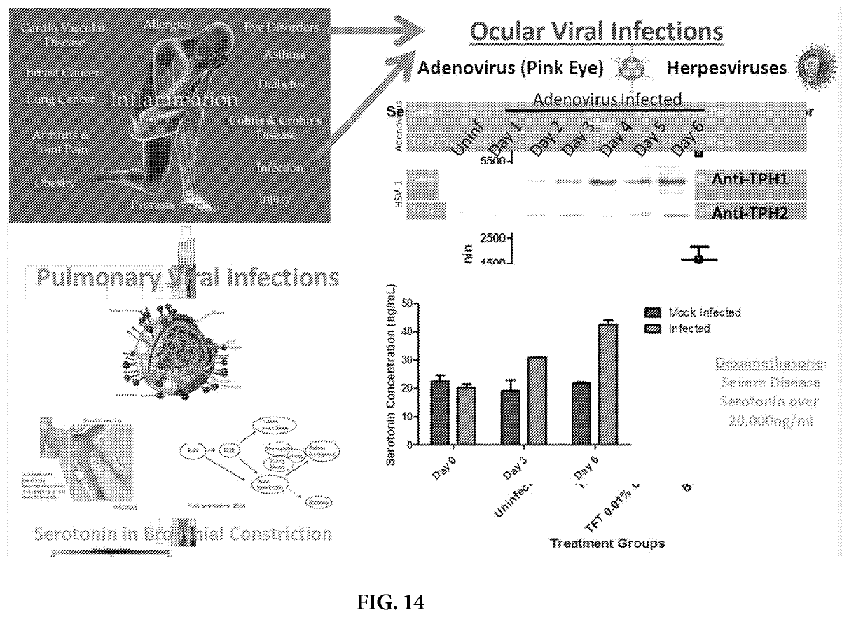

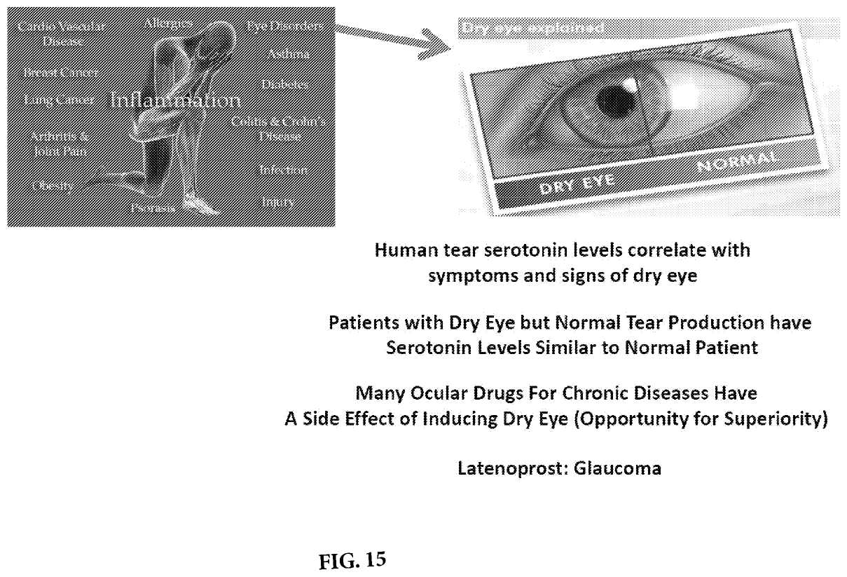

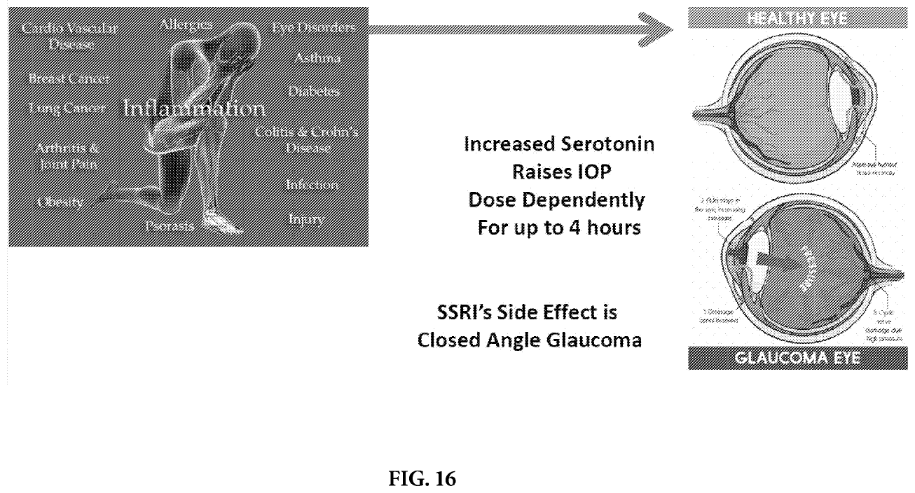

Vascular Effects
Immunological Effects
Fibrosis Effects
Neuroprotective Effects
Information can be extrapolated to other indications
Superiority over other standard treatments
(Can be superior to steroidal anti-inflammatory)
Therapeutic Targets in pathways to disease
What can we treat with 5-HT2A agonists?
Interrelated Pathophysiology of Disease (FIG. 13)
Serotonin Linkages with Disease (FIG. 14)
   Human tear serotonin levels correlate with symptoms and signs of dry eye (FIG. 15)
   Patients with dry eye but normal tear production have serotonin levels similar to normal patient
   Ocular drugs for chronic diseases have a side effect of inducing dry eye
   Latanoprost: Glaucoma
   Increased Serotonin
   Raises IOP
   Dose Dependently
   For up to 4 hours
   SSRI's side effect is closed angle glaucoma (see, for example, FIG. 16)

### 5-HT2AR Agonists as Ocular Therapeutics:

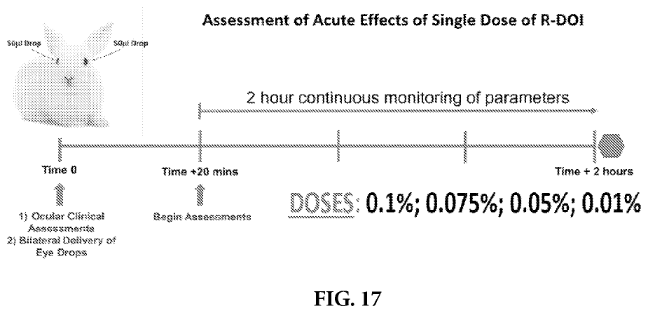

### Topical Ocular Delivery Safety and Tolerability (See, for Example, FIG. 17):

observable behavioral toxicity concentration
ocular tolerability and irritation
direct corneal epithelial cytotoxicity
   0.075% (Approx. 25 μg/Kg):
Tolerable Ocular Delivery; No overt acute signs of irritation
No behavioral changes
Other Ocular Observations: Slight Miosis (Contraction of Pupil)
   0.05% (Approx. 17.5 μg/kg):
Tolerable Ocular Delivery; No Overt Acute Signs of Irritation
No behavioral changes

### Other Ocular Observations: Non

### Observed Parameters:

### CNS:

head twitch
head bob
spasms
listing

### Systemic:

relative increased heart rate
relative increased respiration
short-term weight change

### Ocular:

pupil dilation/contraction
epiphora
intraocular pressure

### Behavioral:

grooming
response to auditory stimuli
lethargy
disorientation
aggression

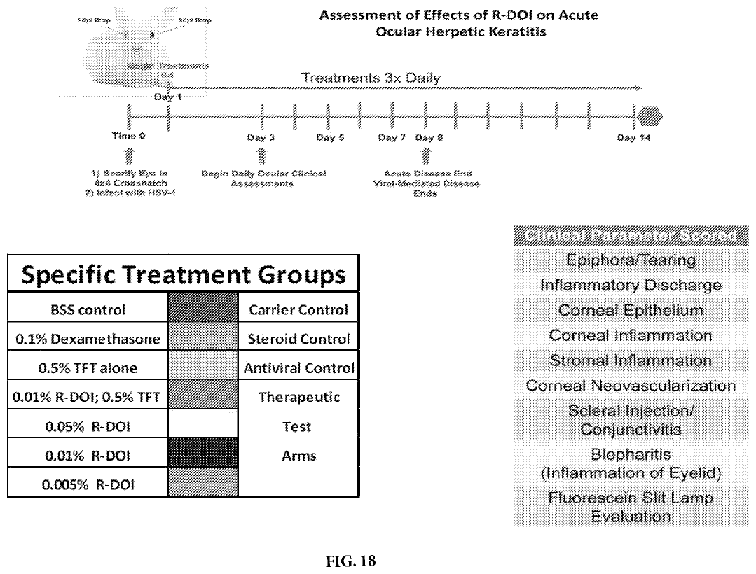

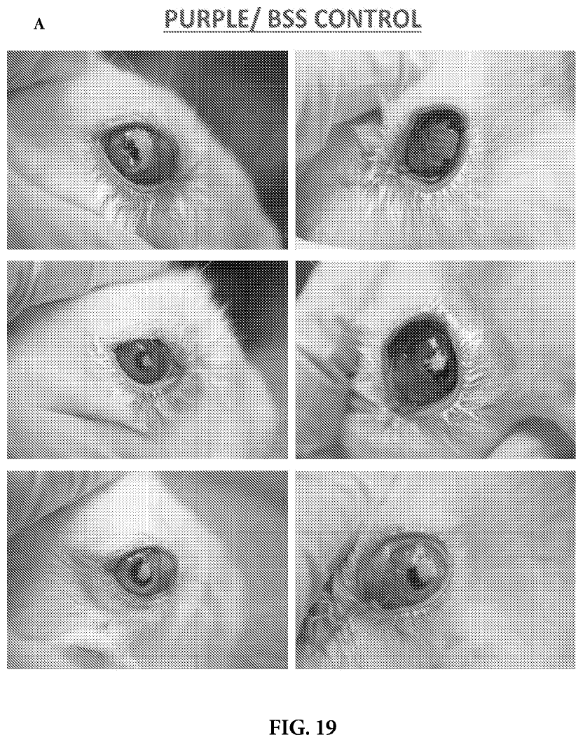

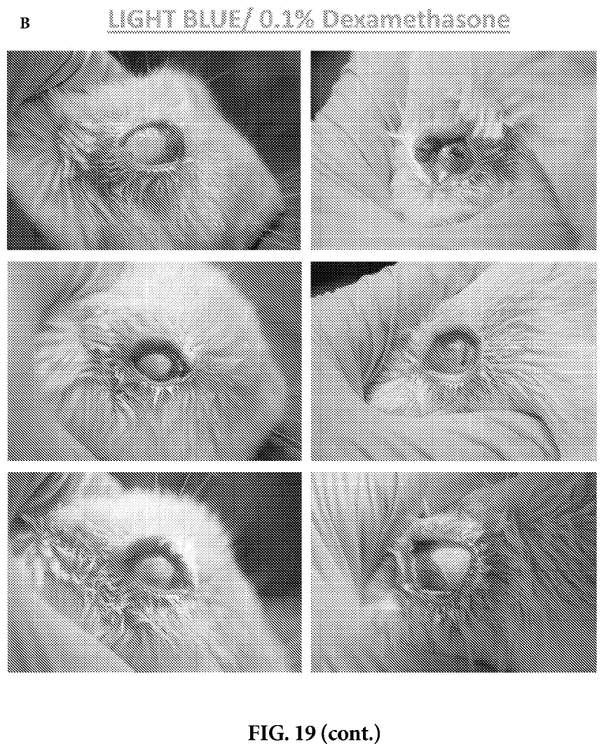

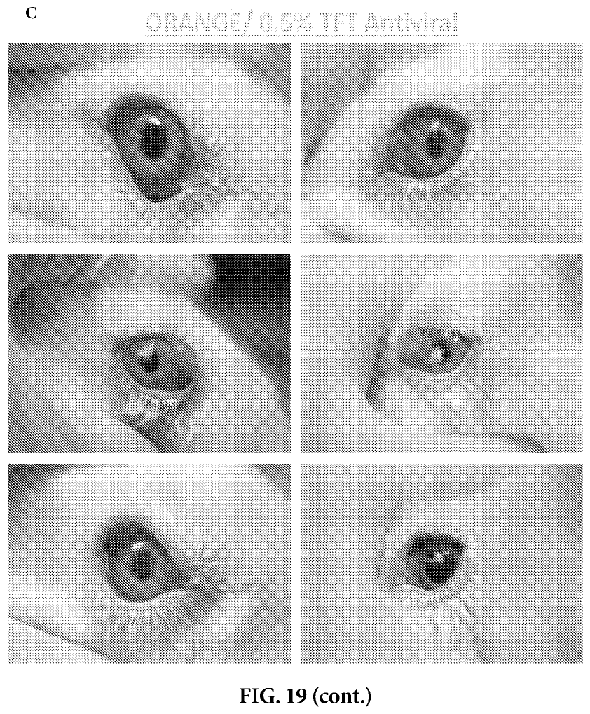

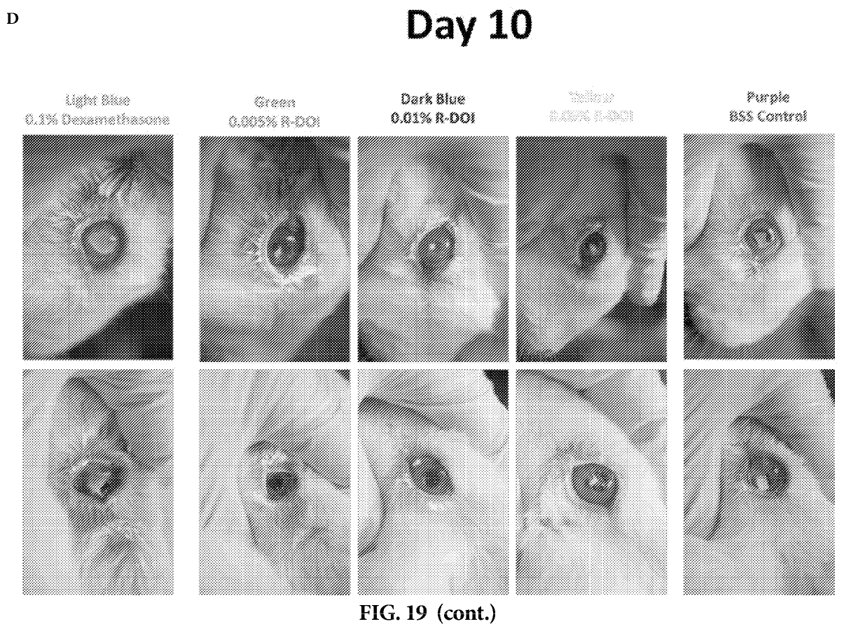

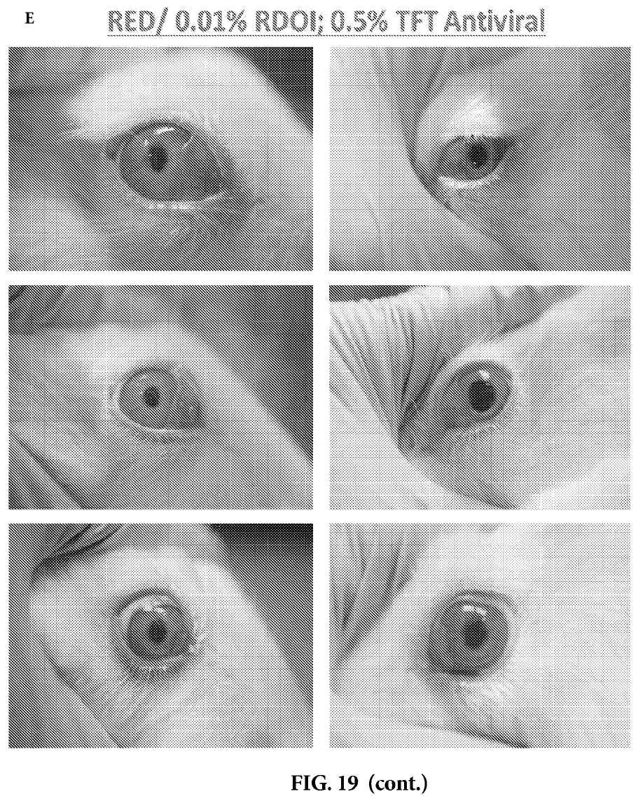

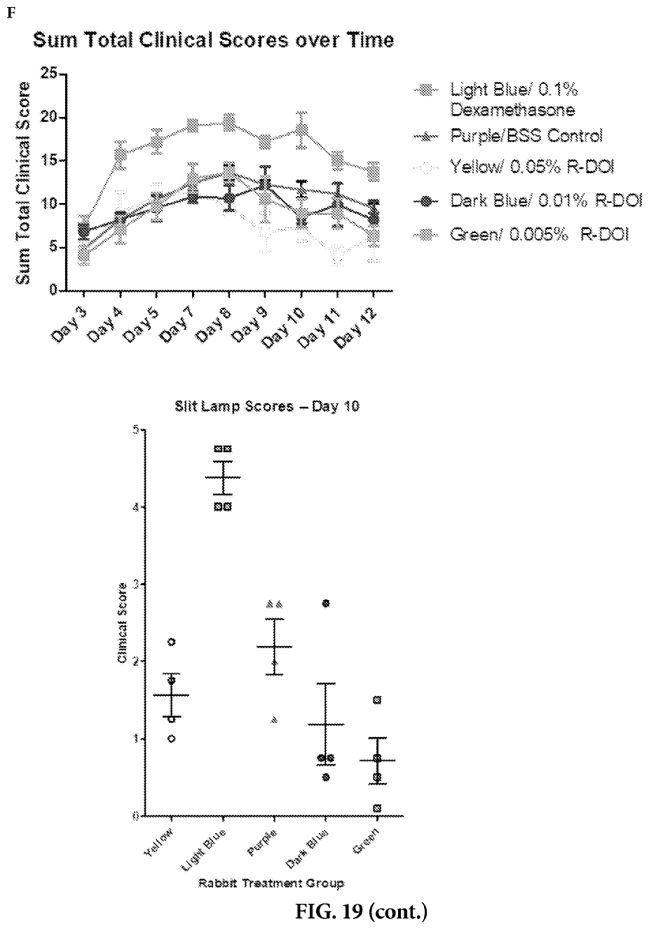

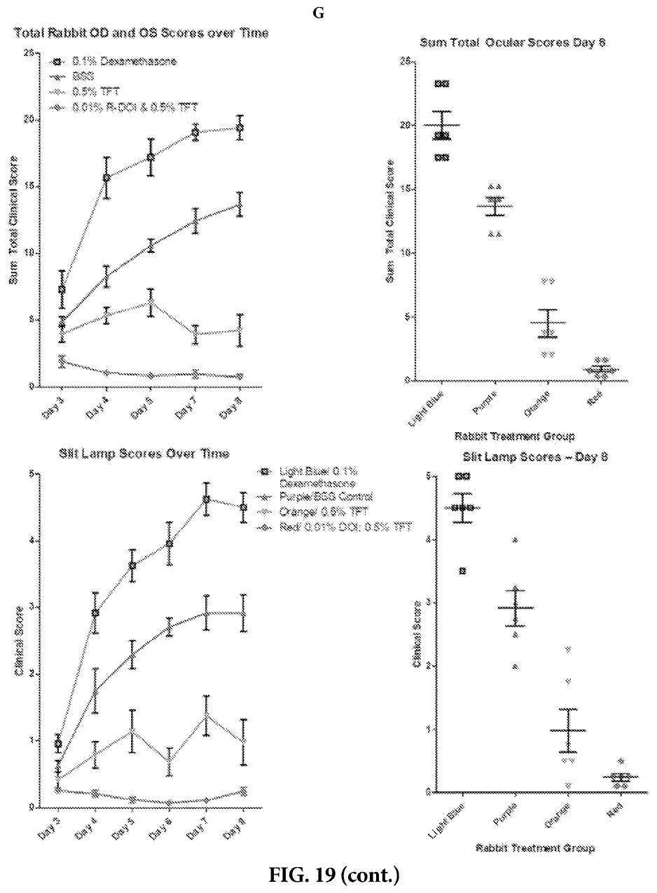

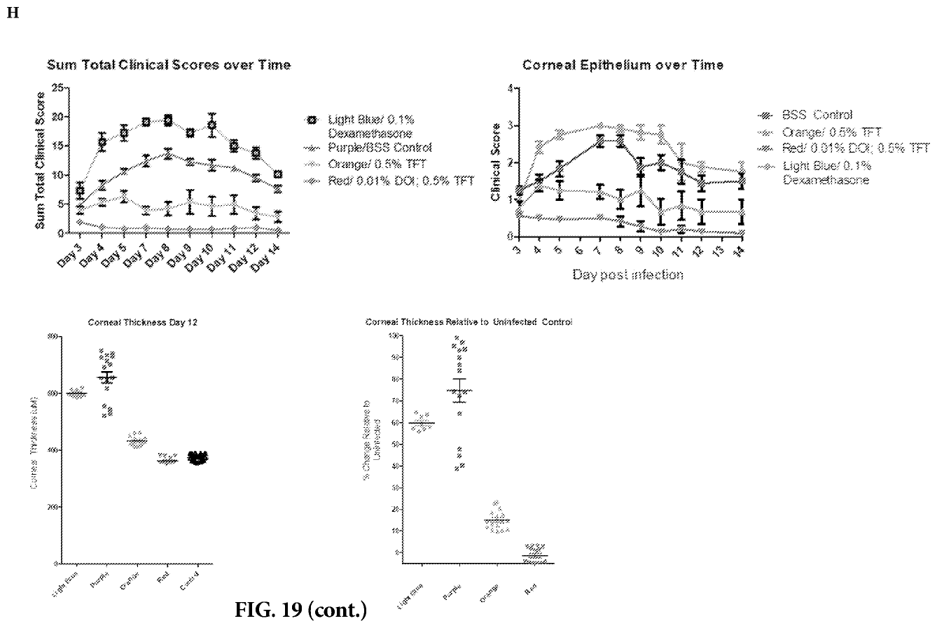

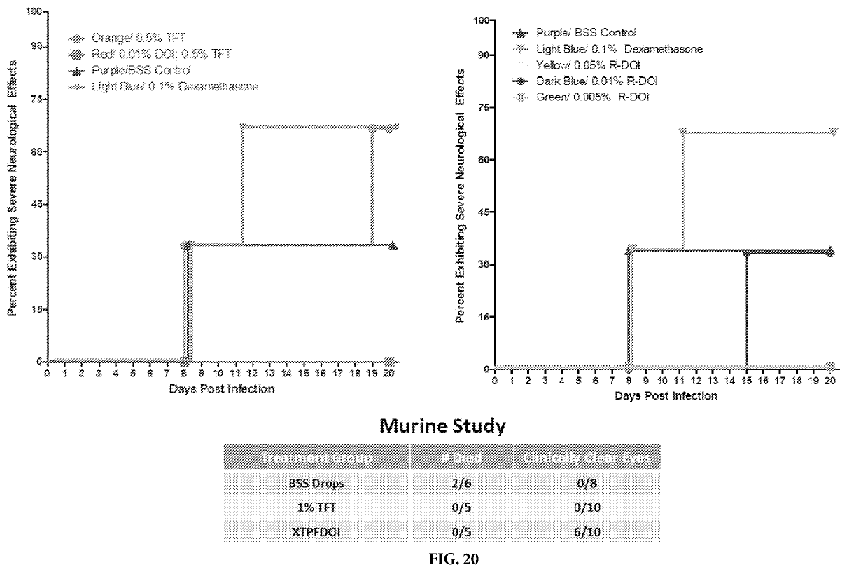

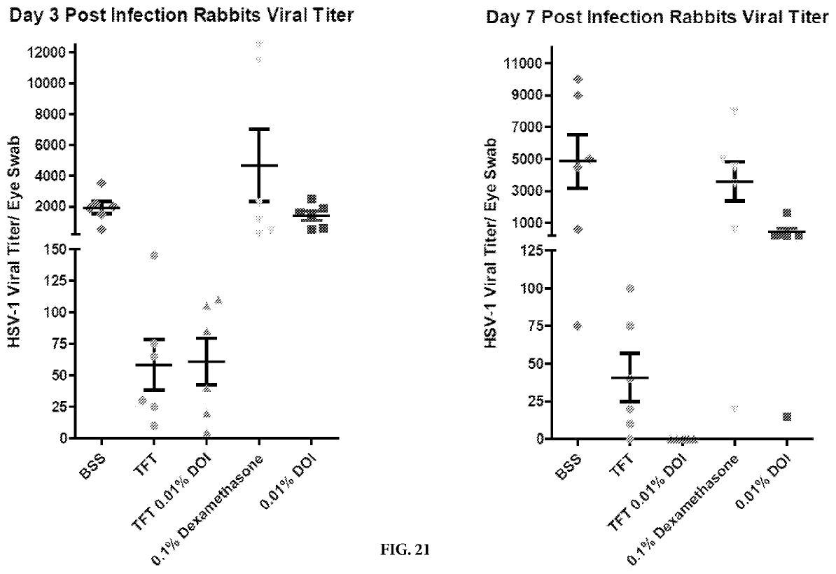

Efficacy in Ocular Models (see, for example, FIGS. 18-21): Acute Herpetic Eye Disease

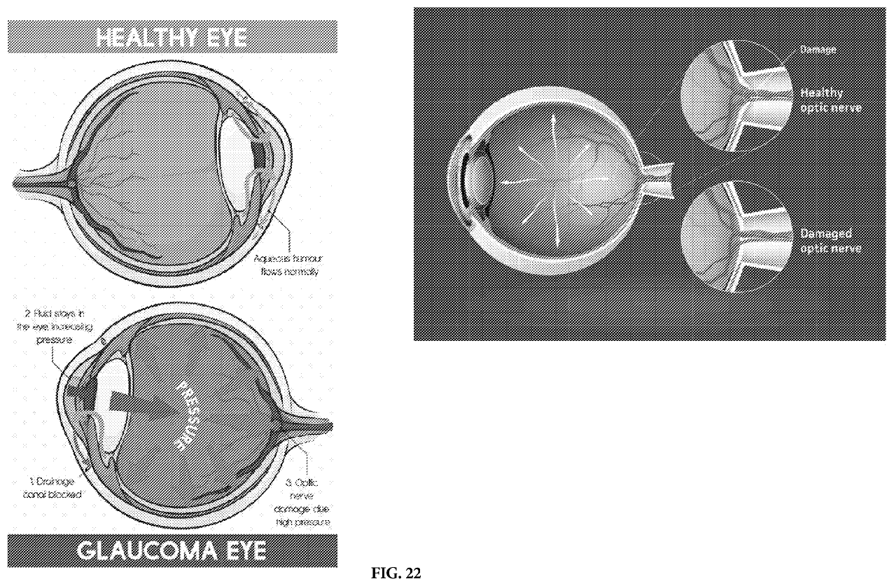

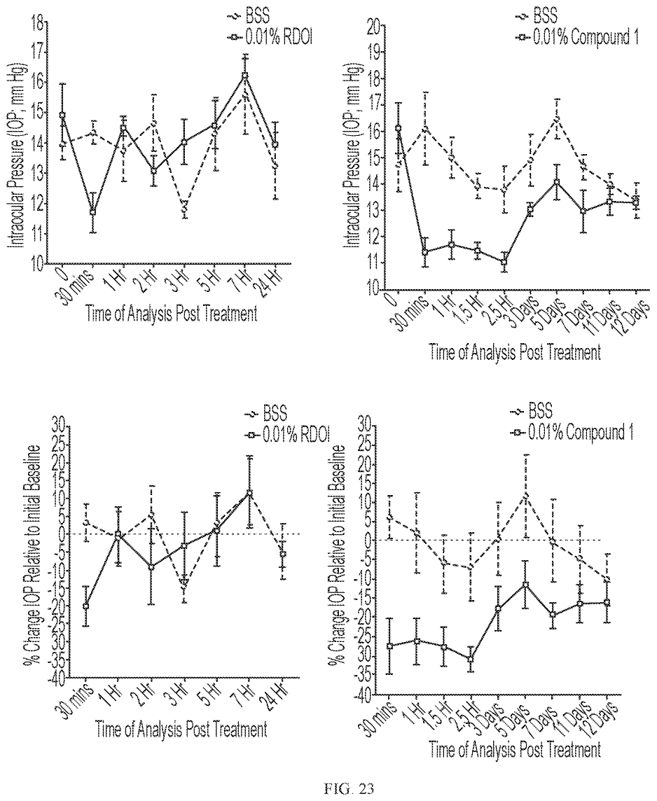

Efficacy in Ocular Models (see, for example, FIGS. 22-24): Ocular Hypertension

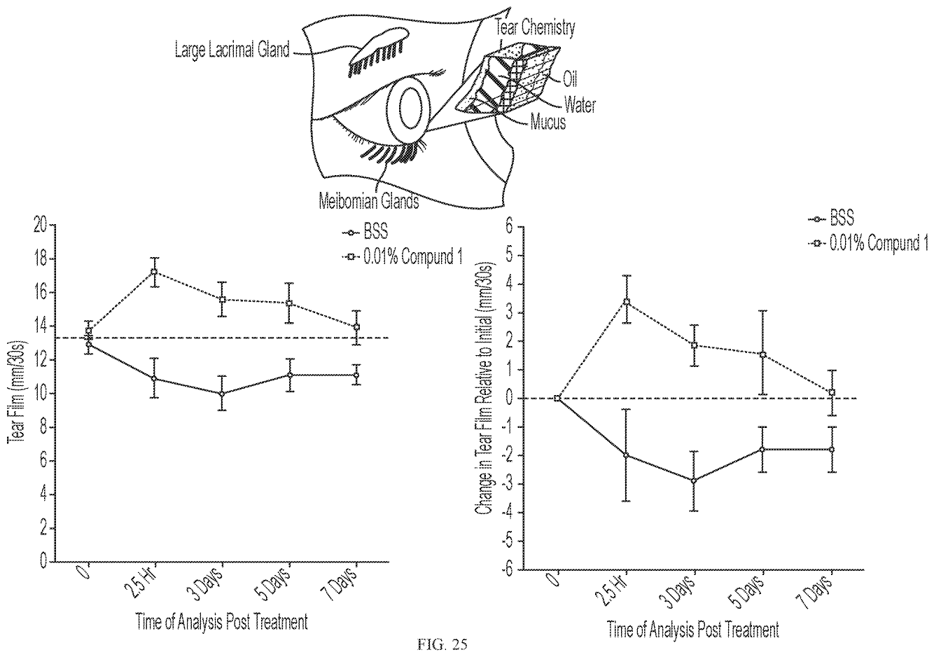

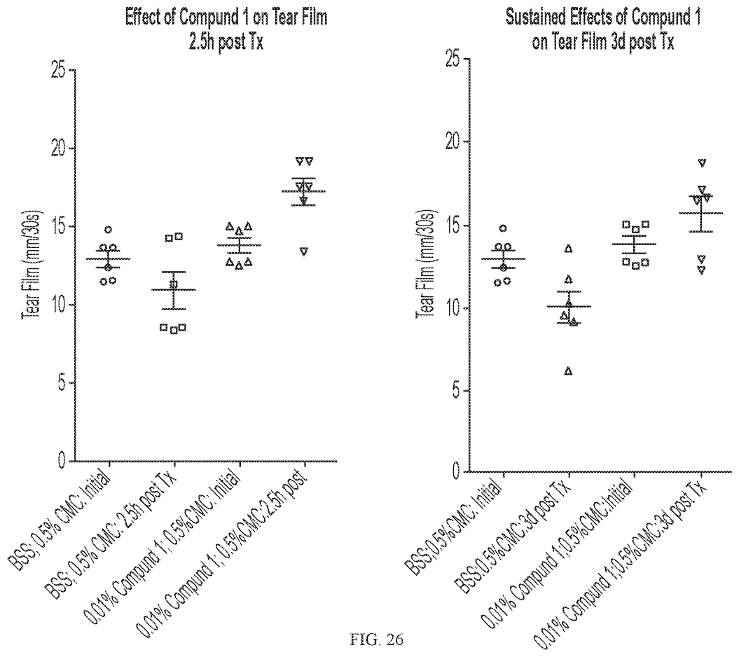

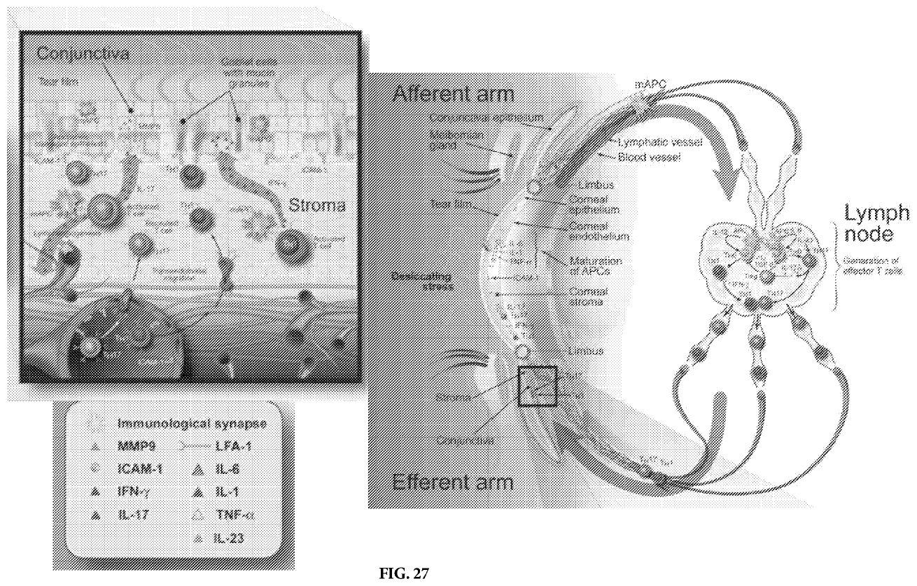

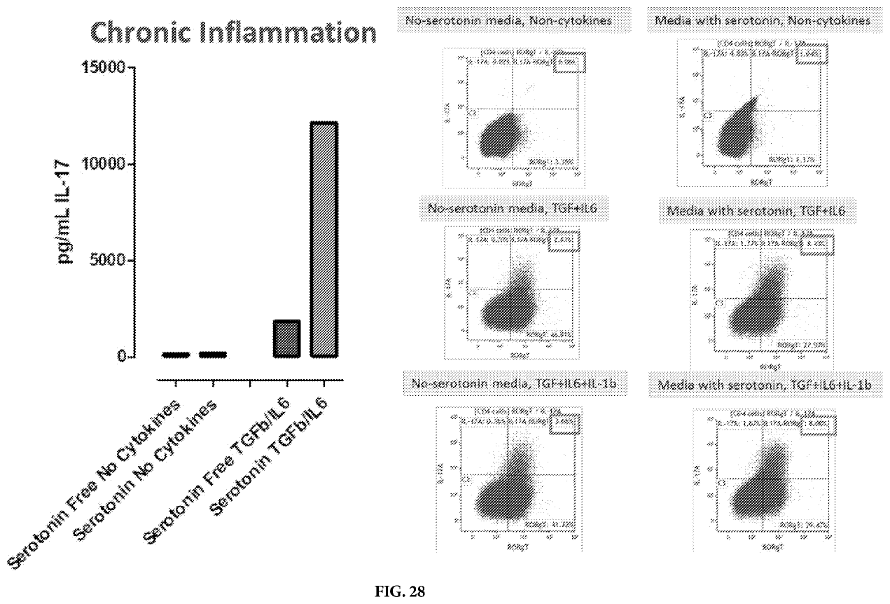

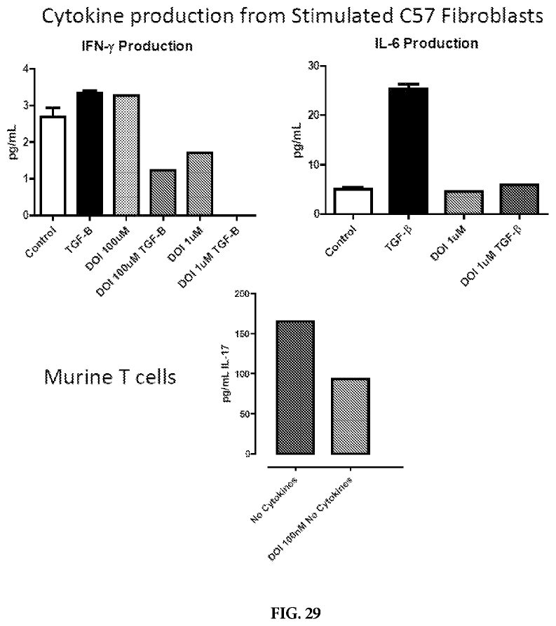

Efficacy in Ocular Models (see, for example, FIGS. 25-29): Increased Tear Production

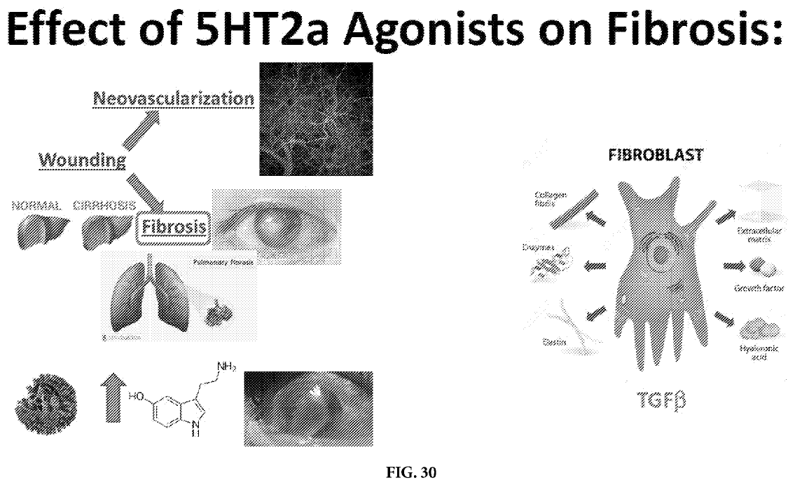

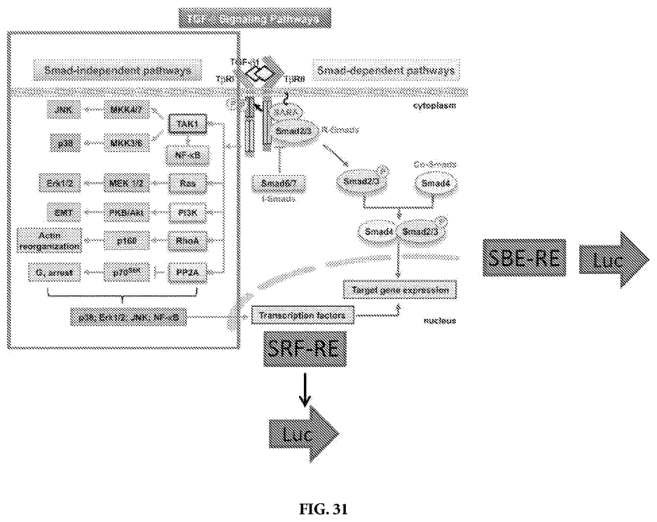

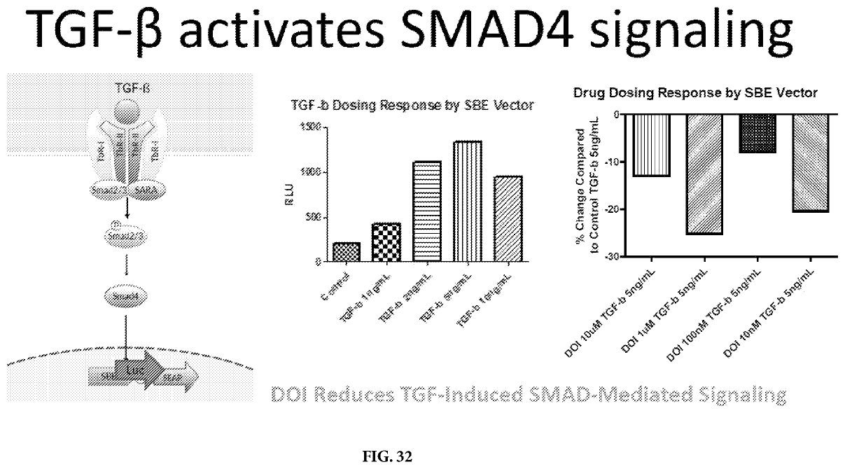

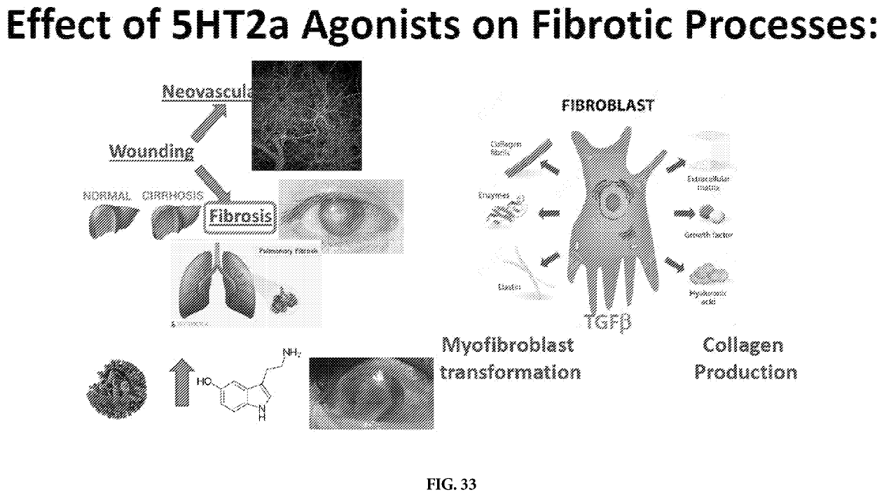

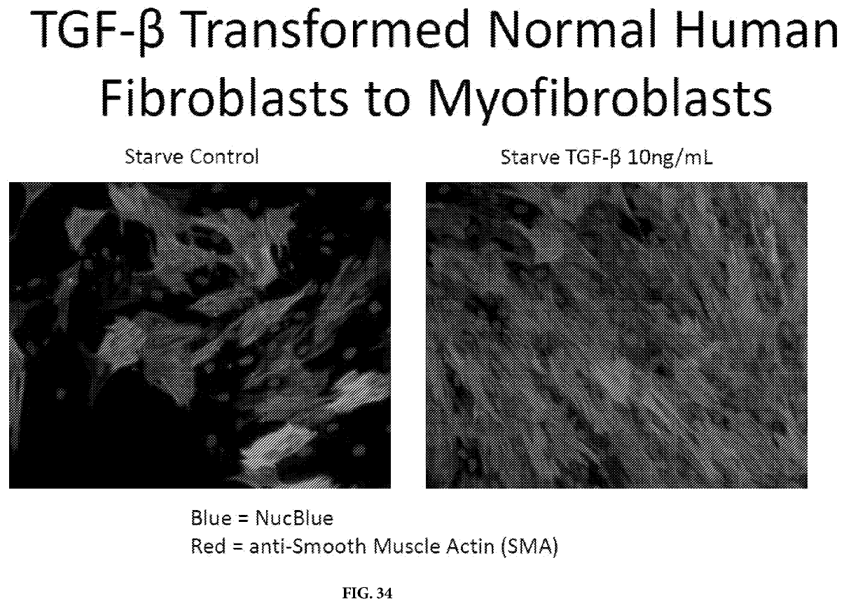

Effect of 5-HT2A Agonists on Fibrosis (see, for example, FIG. 30):
TGF-β Activates Dual Luciferase Reporters (see, for example, FIGS. 31-33)
TGF-β Activates SMAD4 signaling (see, for example, FIG. 32)
   DOI Reduces TGF-Induced SMAD-mediated signaling
TGF-β Transformed Normal Human Fibroblasts to Myofibroblasts (see, for example, FIG. 34)

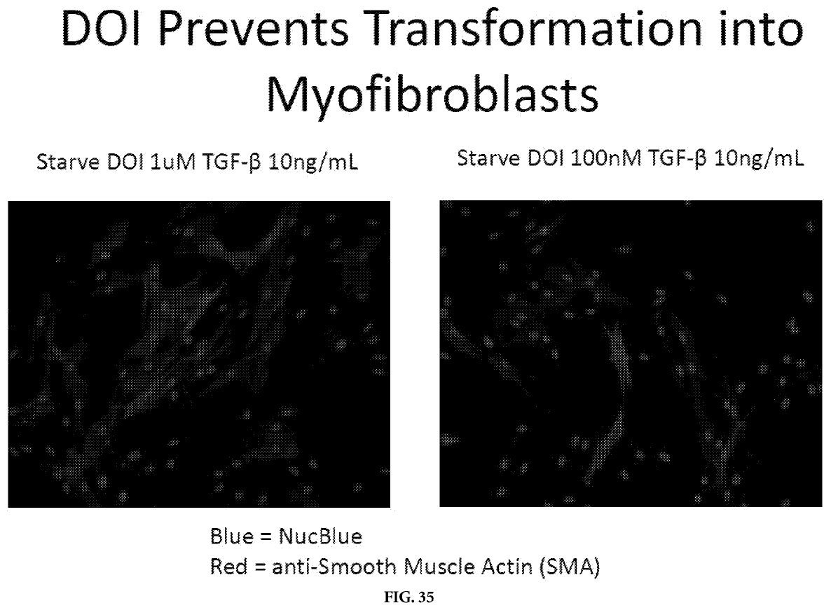

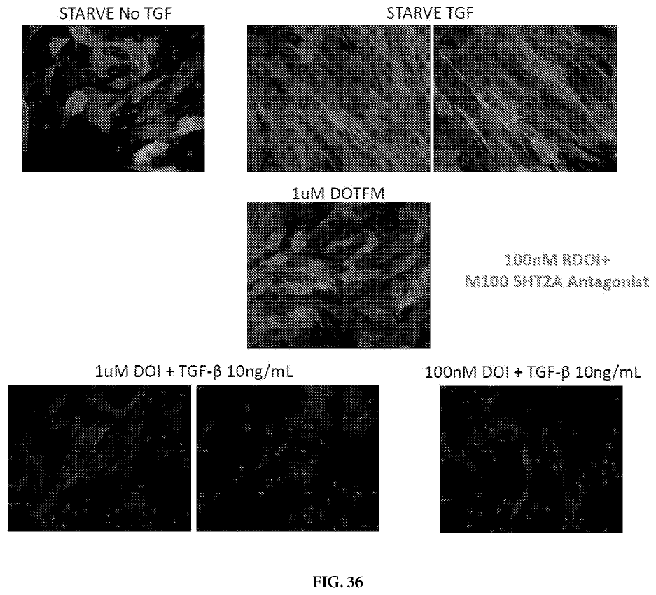

DOI Prevents Transformation into Myofibroblasts (see, for example, FIGS. 35-36)

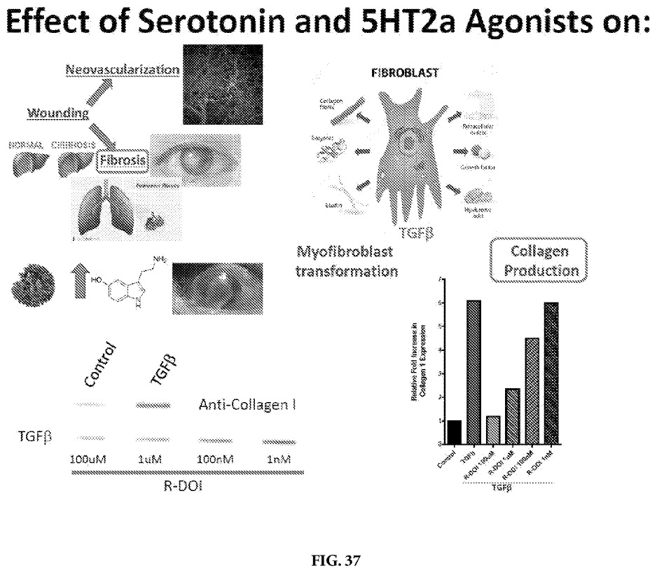

Effect of Serotonin and 5-HT2A Agonist on (see, for example, FIG. 37):

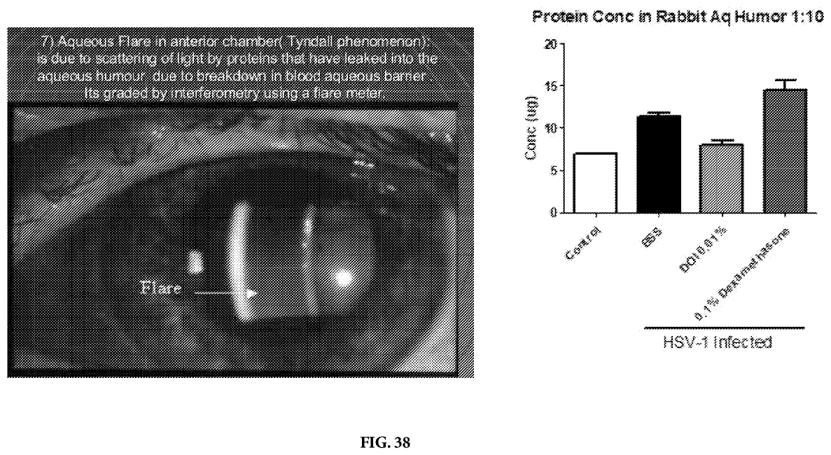

Anterior Chamber Flare (see, for example, FIG. 38):
Chromatic Aberration due to AH protein concentration
Increased AH protein concentration often due to inflammatory protein secretion
Also includes extracellular matrix proteins by myofibroblasts

### In Some Embodiments, the Compositions Described Herein can be Useful for Veterinary Equine Uveitis

Speaking with the Vet Ophthalmologists:
We need to show vitreal haze and anterior flare effects
Equines have conjunctival effects, something not prevalent in Tb model
Concern over Miosis—This is the Pain Side in Horse

### Data Presented on Intravitreal Shows Promise

Can consider composition for delivery

### Serotonin has Effects on Viral Infections—

Ocular Adenovirus-More Conjunctival Effect
Lack of Treatment; Disease is Truly Immune-Mediated
Issue with Steroids

### Effect of Compound 1 NCE on Ocular Diseases

What is best model for fun: IOP; Tear Film; etc.
What can we obtain from it?

Effects of 5-HT2A Agonists on other Aspects of Herpetic Eye

Using in vitro assays to try to understand dosing curve/true receptor targeting, etc.

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed:

1. A method of treating a 5-HT2-mediated ocular condition in a subject in need thereof, the method comprising administering less often than once daily to an eye of the subject compound 1: 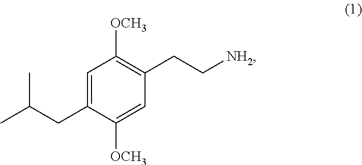 [SMILES: COc1cc(CC(C)C)c(OC)cc1CCN]
or a pharmaceutically acceptable salt thereof, in an amount effective to treat the ocular condition.

2. The method of claim 1, wherein the 5-HT2-mediated ocular condition is a 5-HT2A-mediated ocular condition.

3. The method of claim 1, wherein the ocular condition is associated with decreased lacrimation or tear film production.

4. The method of claim 3, wherein the ocular condition is dry eye.

5. The method of claim 1, wherein the ocular condition is associated with increased intraocular pressure (IOP).

6. The method of claim 5, wherein the ocular condition is glaucoma.

7. The method of any one of claims 1-6, wherein the ocular condition is the result of a side effect associated with another treatment undertaken by the subject.

8. The method of claim 7, wherein the ocular condition is decreased lacrimation or tear film production in a subject undergoing treatment with prostaglandins, alpha-adrenergic agonists, beta-adrenergic blocking agents, rho kinase inhibitors, or carbonic anhydrase inhibitors.

9. The method of claim 1, wherein the ocular condition is IOP in a subject undergoing treatment with an ocular steroid.

10. The method of claim 1, wherein the ocular condition is associated with inflammation.

11. The method of claim 10, wherein the ocular condition is conjunctivitis or keratitis.

12. The method of claim 1, wherein the ocular condition is associated with pathological ocular neovascularization.

13. The method of claim 12, wherein the pathological ocular neovascularization is a corneal neovascularization or a choroidal neovascularization.

14. The method of claim 12 or 13, wherein the pathogenic ocular neovascularization is associated with macular degeneration, keratoconjunctivitis, conjunctivitis, diabetic retinitis, retinopathy of prematurity, polypoidal choroidal vasculopathy, ischemic proliferative retinopathy, retinitis pigmentosa, cone dystrophy, proliferative vitreoretinopathy, retinal artery occlusion, retinal vein occlusion, Leber's disease, retinal detachment, retinal pigment epithelial detachment, rubeosis iridis, corneal neovascularization, retinal neovascularization, choroidal neovascularization, retinochoroidal neovascularization, cancer, or a combination thereof.

15. The method of claim 14, wherein the ocular condition is macular degeneration.

16. The method of claim 1, wherein the ocular condition is associated with scarring of the eye.

17. The method of any one of claims 1-16, wherein the administering is topical administration, instillation in the conjunctival sac, intravitreal administration, subconjunctival administration, retrobulbar, intracameral, or sub-Tenon's administration.

18. The method of claim 17, wherein the administering is by eye drop or gel.

19. The method of any one of claims 1-18, wherein compound 1, or a pharmaceutically acceptable salt thereof, is administered once every other day to once monthly to the eye of the subject.

20. The method of claim 19, wherein compound 1, or a pharmaceutically acceptable salt thereof, is administered once every 2 days, 3 days, 4 days, 5 days, 6 days, 7 days, 8 days, 9 days, 10 days, 11 days, 12 days, 13 days, or 14 days to the eye of the subject.

21. The method of any one of claims 1-20, wherein an eye drop or instillation comprising from about 0.005% to about 0.15% (w/w) of the compound 1, or a pharmaceutically acceptable salt thereof, is administered to the subject.

22. An aqueous pharmaceutical composition suitable for ocular administration comprising:
(i) from about 0.005% to about 0.15% (w/w) of compound 1: 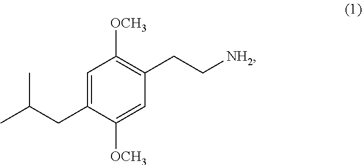 [SMILES: COc1cc(CC(C)C)c(OC)cc1CCN]
or a pharmaceutically acceptable salt thereof; and
(ii) a buffer,
wherein the aqueous pharmaceutical composition has a pH of between 5.0 and 7.0.

23. The aqueous pharmaceutical composition of claim 22, wherein the buffer is a phosphate, borate, or citrate buffer.

24. The aqueous pharmaceutical composition of claim 22 or 23, further comprising a viscosity enhancer.

25. The aqueous pharmaceutical composition of any one of claims 22-24, wherein the aqueous pharmaceutical composition is free of preservatives.
